SEQUENCE LISTING (10 sequences)

SEQ ID NO: 1 - DNA, 59 bp, synthetic construct
  source 1..59: mol_type = other DNA
tttttggtgacgatcccgcaaaatccaatgatgagcactttttgcaagcctcagcgacc

SEQ ID NO: 2 - DNA, 56 bp, synthetic construct
  source 1..56: mol_type = other DNA
tttttggtgacgatcccgcaaaagcggcctttaactccctgcaagcctcagcgacc

SEQ ID NO: 3 - DNA, 56 bp, synthetic construct
  source 1..56: mol_type = other DNA
tttttggtgacgatcccgcaaaatccctcctagttcccctgcaagcctcagcgacc

SEQ ID NO: 4 - DNA, 56 bp, synthetic construct
  source 1..56: mol_type = other DNA
tttttggtgacgatcccgcaaaatcacctaccggcctcctgcaagcctcagcgacc

SEQ ID NO: 5 - DNA, 18 bp, synthetic construct
  source 1..18: mol_type = other DNA
ggtgacgatcccgcaaaa

SEQ ID NO: 6 - DNA, 17 bp, synthetic construct
  source 1..17: mol_type = other DNA
ggtcgctgaggcttgca

SEQ ID NO: 7 - DNA, 17 bp, synthetic construct
  source 1..17: mol_type = other DNA
caatgatgagcactttt

SEQ ID NO: 8 - DNA, 14 bp, synthetic construct
  source 1..14: mol_type = other DNA
cggcctttaactcc

SEQ ID NO: 9 - DNA, 14 bp, synthetic construct
  source 1..14: mol_type = other DNA
cctcctagttcccc

SEQ ID NO: 10 - DNA, 14 bp, synthetic construct
  source 1..14: mol_type = other DNA
acctaccggcctcc

(19) United States
(12) Patent Application Publication
(10) Pub. No.: US 2026/0235632 A1
(43) Pub. Date: Aug. 13, 2026

(54) ASSAY SYSTEM

(71) Applicant: ACTOME GMBH, Freiburg (DE)

(72) Inventor: Csaba JENEY, Freiburg (DE)

(21) Appl. No.: 19/156,159

(22) PCT Filed: Dec. 7, 2023
(86) PCT No.: PCT/EP2023/084793

§ 371 Date: Aug. 13, 2025

(30) Foreign Application Priority Data
Dec. 8, 2022 (GB) 2218453.5

(51) Int. Cl. G01N 33/74; G01N 33/577

(52) U.S. Cl. CPC G01N 33/74; G01N 33/577; G01N 2333/4704; G01N 2333/71; G01N 2440/14; G01N 2458/10

(57) ABSTRACT

A method of concurrently determining the concentration and/or status of an analyte using pairwise readout of three or more binding agents, where each of said binding agents binds to a different site on the analyte is described. The amount of analyte present is determined by detecting the presence of two or more pairs of binding agents using a bi-component detection method.

The invention relates to a method of concurrently determining the concentration and/or status of an analyte using pairwise readout of three or more binding agents, where each of said binding agents binds to a different site on the analyte. The amount of analyte present is determined by detecting the presence of two or more pairs of binding agents using a bi-component detection method.

### Protein Interaction Coupling

A protein interaction coupling assay uses a pair of binding agents, each associated with a unique label, directed against a target. The complex formed by a target with two bound binding agents is referred to herein as a ‘couplex’. The couplex is the molecular unit which is detected by the assay by counting each couplex individually. The two binding agents, directed against two non-overlapping epitopes, can detect a target which can be used to investigate characteristics of proteins, protein interactions and post-translational modification of proteins. Even less specific binding agents (such as anti-phospho and polyclonal antibodies) can be used as the two binding agent design inherently has a high specificity. The assay can be used for measuring the same analyte independently by more than two binding agents per target as well as quantifying analytes such as proteins, and investigating their interactions and modifications in the same reaction. Experimenters can use almost any binding agent pair, such as antibodies including polyclonal antibodies in an interaction coupling assay. As the assay specificity is inherently very high, there is no requirement to use pairs of binding agents which have been matched by species or specificity. The only requirement is that the binding agents have two distinct epitopes so can bind to the target concurrently and that in case of interaction detection that their epitopes are not masked by other interacting partners.

A protein interaction coupling assay capable of measuring analytes in absolute quantities is described in WO2020260277A1. Briefly, as protein interaction coupling counts both antibodies and couplexes molecularly, having a detection unit of molecules per reaction, the molar concentration of these analytes can be calculated from the experimental conditions including volumes and dilution, and optionally binding constants of antibodies. According to WO2020260277A1 knowing the dissociation constant of the antibodies involved (or its derivative for polyclonal antibodies), and using chemical theoretical considerations including mass conservation and chemical balances, the absolute amount of the target analytes can be calculated.

The present invention extends these concepts further by introducing absolute homogeneous internal multiplexing (AIM). Absolute homogeneous internal multiplexing exploits the discovery that more than one binding agent can bind to a given target and a bi-component immunoassay can read the absolute quantities of these binding agents pairwise enabling an array of confirmatory and conditional absolute quantitative measurements.

The present invention provides examples of detection assays using three or four or even more binding agents, such as antibodies per target analyte. As exemplified, the methods of the invention are useful to measure the phosphorylated fraction of a given protein using two antibodies capable of binding to different epitopes on the protein and a third antibody which specifically binds to the phosphorylated form of the protein, enabling measurement of the absolute amount of the protein and two different ways of measuring the absolute amount of the phosphorylated protein. Similarly, protein interactions using four antibodies, specifically two pairs of antibodies against each protein of a pair of interacting protein partners, can be used measure the absolute amount of each of the two interacting proteins, and four different antibody pairs provide measurements for the interactions between them. Adding a fifth antibody into the system against a phosphorylated variant of the interacting proteins can extend the confirmatory measurements and even enables conditional measurements where the detection of phosphorylation is conditional on the protein interaction.

Thus in a first aspect the invention provides a method of determining the concentration and/or status of an analyte comprising:
(a) contacting the analyte with three or more binding agents, wherein each of said binding agents binds to a different site on the analyte and is associated with a unique label;
(b) detecting the amount of analyte bound by two or more pairs of binding agents using a compartmentalised bi-component detection method;
(c) determining the concentration and/or status of the analyte present based on the measurements obtain in step (b).

Preferably, at least one of the binding agents binds specifically to an unmodified or modified analyte. Preferably the analyte is modified by phosphorylation, methylation, sulfation, acetylation, ubiquitylation, prenylation, myristoylation, sumoylation, palmitoylation, different types of glycosylation (N-glycosylation, O-glycosylation, C-glycosylation and S-glycosylation), phosphoglycosylation, glycosylphosphatidylinositol (GPI anchored), methylation or other known amino acid or protein modification.

The analyte is preferably a protein, peptide, nucleic acid, carbohydrate, lipid, microorganism or fragment thereof, cell, or complex or combinations thereof.

The invention also provides kit for determining the concentration and/or status of an analyte comprising:
(a) three or more binding agents, wherein each of said binding agents binds to a different site on the analyte and is associated with a unique label suitable for use in bi-component detection method; and
(b) reagents for detecting pairs of binding agents using a bi-component detection method; and/or
(c) a computer programme which when run on a computer analyses the results of the bi-component detection method and determines the concentration of the analyte in a sample; and optionally
(d) instructions for carrying out the bi-component detection method.

Each binding agent has a label which is unique to the binding agent. The label can be a nucleotide sequence, preferably an oligonucleotide, or a fluorescent dye.

Preferably the reagents in the kit for detecting pairs of binding agents using a bi-component detection method, wherein the binding agents are each labelled with a unique oligonucleotide sequence, comprises
(i) a forward primer and reverse primer for amplifying the unique oligonucleotide sequences; and
(ii) three or more oligonucleotide probes, where each oligonucleotide probe is independently capable of specifically binding to a unique oligonucleotide sequence amplified by the primers and independently labelled.

### DETAILED DESCRIPTION

As used herein, the term “nucleic acid amplification” refers to a process by which a limited quantity of nucleic acid undergoes a biochemical reaction in which a larger quantity of nucleic acid is generated. Nucleic acid amplification thus relates to the generation of additional copies of a nucleic acid sequence and is generally carried out using polymerase chain reaction (PCR) or ligase chain reaction (LCR) or other technologies well known in the art (see, for example, Dieffenbach, C. W. and G. S. Dveksler (1995) PCR Primer, a Laboratory Manual, Cold Spring Harbor Press, Plainview, N.Y.).

As used, herein the term “bi-component detection method” refers to a method using analyte-specific binding agents and detecting a signal reflecting the presence of two analyte-specific binding agents following the formation of binding agents/analyte complexes containing the two binding agents and the analyte e.g. the “couplex”, when the analyte-specific binding agents are brought into contact with a solution or sample containing the analyte.

The term “bi-component detection system”, refers to components or reagents necessary to carry out the bi-component detection method. This encompasses the analyte-specific binding agents, preferably provided in a single or separate solution at a known concentration as well as an optional a sample solution containing the analyte to be analyzed.

Preferably the analyte-specific binding agents and/or the analyte are provided in a non-immobilized form, i.e. in solution, such that the couplexes (analyte/binding agent complexes) likewise form in solution. The bi-component detection methods as used herein, thus preferably encompass formation of ternary complexes in a liquid phase and are distinct from common sandwich immunoassays involving as a solid phase immobilized (primary) capture binding agents as well (secondary) detection binding agents. The term “non-immobilized” thus preferably refers to components, such as the analyte-specific binding agents, that may freely diffuse within a liquid, such that the kinetics of binding to the analyte, which is equally freely diffusing in said liquid, are governed by equations for law of conservation of mass and law-of-mass action in solution.

The term “binding agents” or “analyte specific-binding partners” refers to one member of a binding pair, wherein the second member is the analyte. The term “binding pair” includes any of the class of immune-type binding pairs, such as antigen/antibody or hapten/anti-hapten systems; and also any of the class of nonimmune-type binding pairs, such as biotin/avidin; biotin/streptavidin; folic acid/folate binding protein; complementary nucleic acid segments such as complementary DNA strands or complementary RNA strands; protein A or G/immunoglobulins; and binding pairs which form covalent bonds, such as sulfhydryl reactive groups including maleimides and haloacetyl derivatives, and amine reactive groups such as isotriocyanates, succinimidyl esters and sulfonyl halides.

The binding agents of the bi-component detection method may refer to any component having a binding potential to the analyte. In preferred embodiments the analyte-specific binding agents may be nucleic acids, preferably RNA and/or DNA oligonucleotides, antibodies, peptides, proteins, aptamers, molecularly-imprinted polymers, cells or combinations of thereof. Preferably at least one of the binding agents is an antibody, more preferably a monoclonal antibody.

As used herein, the term “antibody” is used interchangeably with “immunoglobulin” and encompasses polyclonal antibodies, monoclonal antibodies, multispecific antibodies such as bispecific antibodies, chimeric antibodies, humanized antibodies, human antibodies, and any other modified immunoglobulin molecule comprising an antigen recognition site so long as the antibodies exhibit the desired biological activity, and contain at least the CH2 portion of at least one heavy chain. Preferably the antibody is a monoclonal antibody. An antibody can be a member of any of the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Preferably, the antibody is an IgG antibody, more preferably an IgG1 or IgG4. The term “antibody” is also intended to include single domain antibodies (sdAb) or nanobodies including V_{H}H fragments and V_{NAR} fragments, which consist of a single monomeric antibody chain, but are still able to selectively bind a specific antigen. The term “antibody” is also intended to include conjugates of the antibody, for example conjugates with polyethylene glycol, PEG.

Further, except where the context requires otherwise, the term “antibody” should be understood to encompass complete antibodies and antibody fragments comprising an antigen-binding region of the complete antibody and the CH2 domain, including scFv-CH2-CH3 fusion proteins. An antibody can be produced by a hybridoma, or by synthetic means such as recombinant DNA techniques, phage display or yeast display technologies or using transgenic mice, or liquid or solid phase peptide synthesis.

Complementarity determining regions (CDRs) are part of the variable chains in immunoglobulins (antibodies), generated by B-cells, where these molecules bind to their specific antigen. As the most variable parts of the molecules, CDRs are crucial to the diversity of antigen specificities generated by immunoglobulins. There are three CDRs (CDR1, CDR2 and CDR3), arranged non-consecutively, in the amino acid sequence of a variable domain of an immunoglobulin. Since the immunoglobulins are typically composed of two variable domains (on two different polypeptide chains, heavy and light chain), there are six CDRs for each antigen receptor that can collectively come into contact with the antigen.

It has been shown that fragments of a whole antibody can perform the function of binding antigens. Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (Ward, E. S. et al., Nature 341:544-546 (1989)) which consists of a VH domain; (v) isolated CDR regions; (vi) F(ab′) 2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., Science 242:423-426 (1988); Huston et al., PNAS USA 85:5879-5883 (1988)); (viii) bispecific single chain Fv dimers (PCT/US92/09965) and (ix) “diabodies”, multivalent or multispecific fragments constructed by gene fusion (WO94/13804; P. Hollinger et al., Proc. Natl. Acad. Sci. USA 90:6444-6448 (1993)).

An “antigen binding domain” is the part of an antibody which comprises the area which specifically binds to and is complementary to part or all of an antigen. Where an antigen is large, an antibody may only bind to a particular part of the antigen, which part is termed an epitope. An antigen binding domain may be provided by one or more antibody variable domains. An antigen binding domain may comprise an antibody light chain variable region (VL) and an antibody heavy chain variable region (VH).

Alternatively, the binding agents may be based on engineered protein scaffolds. Protein scaffolds are derived from stable, soluble, natural protein structures which have been modified to provide a binding site for a target molecule of interest. Examples of engineered protein scaffolds include, but are not limited to, affibodies, which are based on the Z-domain of staphylococcal protein A that provides a binding interface on two of its a-helices (Nygren, P. A. (2008). FEBS J 275 (11): 2668-76); anticalins, derived from lipocalins, that incorporate binding sites for small ligands at the open end of a beta-barrel fold (Skerra, A. (2008) FEBS J 275 (11): 2677-83), nanobodies, and DARPins. Engineered protein scaffolds are typically targeted to bind the same antigenic proteins as antibodies. Short peptides may also be used to bind a target protein. Phylomers are natural structured peptides derived from bacterial genomes. Such peptides represent a diverse array of protein structural folds and can be used to inhibit/disrupt protein-protein interactions in vivo (Watt, P. M. (2006). Nat Biotechnol 24 (2): 177-83)].

Alternatively, the binding agent may be an aptamer. Aptamers are synthetic oligonucleotides (DNA or RNA) that recognize target molecules with high affinity and specificity through a combination of shape complementarity and non-covalent chemical bonds (Blank & Blind, Current Opin. Chem. Biol., 2005, 9:336-342). These artificial ligands are quite easy to obtain in vitro and can be developed to recognise a large variety of different molecule classes which range from mere ions (e.g. Pb2+, Liu & Lu, 2003. J Am Chem Soc., 125, 6642-6643) to nucleotides, small molecules, proteins, viruses, and cells up to whole organisms (Menger et al., 2006. Handbook of Experimental Pharmacology, 359-373). High binding affinity aptamers have been selected through the well-known SELEX method (Ellington & Szostak, 1990. Nature, 346, 818-822) for the detection of low molecular weight molecules like theophyllin (Jenison et al., 1994. Science, 263, 1425-1429), L-arginine (Geiger et al., 1996. Nucl. Acids Res., 24, 1029-1036), moenomycin (Schuerer et al., 2001. Bioorg. Med. Chem., 92, 2557-2563), 17b-estradiol (Kim et al., 2007. Biosens. Bioelectron., 22, 2525-2531) but also for larger molecules like thrombin (thrombin-binding aptamer: 5′-GGTTGGTGTGGTTGG-3′) (Baldrich et al., Anal Chem. 2004, 76, 23,7053-63), cholera toxin or HIV-1 tat protein, among others (for review see Tombelli et al., 2007, Biomolec Eng., 24, 191-200). Some of the above mentioned aptamers have been used in ELISA-like assays on microplates or on the surface of biosensor transducers (QCM, SPR). An aptamer-modified AuNP colorimetric system has also been developed for the determination of the protein PDGF in a sandwich-based assay (Huang et al., 2005, 77, 5735-5741).

If the binding agent is an aptamer or nucleic acid, then the label may form part of the aptamer/oligonucleotide The nucleotide sequence may be part of the binding component itself e.g. aptamer, or be present within the binding component e.g. nucleic acid within a phage

If the binding agent is not an aptamer or nucleic acid, it can be labelled with a nucleotide sequence, for example an oligonucleotide. The nucleotide sequence is preferably an oligonucleotide and may comprise RNA or DNA, single or double stranded. Nucleotides used to label the binding agent are generally 5-150 bases in length, for example 10-40, or 20-30 bases in length. The nucleotides that form the nucleic acid can be chemically modified to increase the stability of the molecule, to improve its bioavailability or to confer additional activity on it. For example the pyrimidine bases may be modified at the 6 or 8 positions, and purine bases at the 5 position with CH₃ or halogens such as I, Br or CI. Modifications or pyrimidines bases also include N²—H₃, O₆—CH₃, N⁶—CH₃ and N²—CH₃. Modifications at the 2′position are sugar modifications and include typically a NH₂, F or OCH₃ group. Modifications can also include 3′ and 5′ modifications such as capping.

Alternatively modified nucleotides, such as morpholino nucleotides, locked nucleic acids (LNA) and peptide nucleic acids (PNA) can be used. Morpholino oligonucleotides are assembled from different morpholino subunits, each of which contains one of the four genetic bases (adenine, cytosine, guanine, and thymine) linked to a 6-membered morpholine ring. The subunits are joined by non-ionic phosphorodiamidate intersubunit linkages to give a morpholino oligonucleotide. LNA monomers are characterised in that the furanose ring conformation is restricted by a methylene linker that connects the 2′-O position to the 4′-C position. PNA is an analogue of DNA in which the backbone is a pseudopeptide rather than a sugar.

The term “analyte” as used herein refers to a substance to be detected, quantified or otherwise assayed by the method of the present invention. It is a molecule or group of molecules which form a complex with the binding agent. The complex is usually formed under normal physiological conditions of the organism of interest. Typical analytes may include, but are not limited to proteins, peptides, nucleic acid segments, carbohydrates, lipids, antibodies (monoclonal or polyclonal), antigens, oligonucleotides, specific receptor proteins, ligands, molecules, cells, microorganisms, fragments, products and combinations thereof, or any substance for which attachment sites, binding members or receptors (such as antibodies) can be developed. The term “analytes” also includes complexes formed from said entities. For instance, an analyte may refer to an aggregate or complex formed of multiple entities or molecules, e.g. a protein-protein complex, wherein the interaction of the entities/molecules is of interest. Preferably the analyte is a protein or two or more interacting proteins which bind to one another.

The term “protein” as used herein refers to a sequence of amino acids the chain length of which is sufficient to produce the higher levels of tertiary and/or quaternary structure. “Peptides” preferably refer to smaller molecular weight proteins.

A “modified analyte” as used herein refers to an analyte which has undergone any biological modification which includes posttranslational modifications or the presence of any optional molecular residues on the analyte. Preferably the analyte modified by phosphorylation, methylation, sulfation, acetylation, ubiquitylation, prenylation, myristoylation, sumoylation, palmitoylation, different types of glycosylation (N-glycosylation, O-glycosylation, C-glycosylation and S-glycosylation), phosphoglycosylation, glycosylphosphatidylinositol (GPI anchored), methylation and others.

The analyte is generally present in a sample i.e. a solution comprising analytes, preferably at an unknown concentration. Examples of samples include biological fluids such as serum, plasma, urine, tear, cerebrospinal fluid, blood, saliva, cells, cell mixtures, cell culture supernatants, or cell lysates containing one or more biological target molecules. Alternatively, the sample can be generated by recombinant expression methods. Preparation of proteins from specimens can be performed using standard methods known in the art. Furthermore, samples can also further comprise conditioning reagents (e.g., permeabilising reagents) needed to render analytes soluble and accessible to detection and quantification. Such conditioning reagents may be added to the sample at any time before or after carrying out the methods described herein.

The concept of absolute homogeneous internal multiplexing extends the capabilities of measuring biological systems in an absolute quantitative and high content way projecting the description of the biological processes into the chemical and stoichiometric understanding.

Bi-component detection systems are typically exploited in homogeneous assays where two components are applied to produce detection signals. Such detection systems are described in WO2020260277, which is incorporated herein by reference.

The method for determining the concentration of an analyte can use compartmentalized bi-component detection methods which allow a higher magnitude of measurement range. Compartmentalized bi-component detection methods are highly preferred as they allow to determine the absolute concentration of the analyte.

In particular preferred embodiments the methods as described herein uses compartmentalized bicomponent detection methods. As used herein the term “compartmentalized bi-component detection methods” refers to bi-component detection methods that compartmentalize the sample after bringing the analyte specific binding agents in contact with the analyte to quantify the formation of ternary analyte/binding-components.

In general, the compartmentalized bi-component detection methods are based upon separating or compartmentalizing a solution to small compartments such that without the formation of ternary analyte/binding-component methods the presence of both binding agents in the compartment is unlikely and follows a Poisson distribution. In the method, the couplexes are separated and isolated from other couplexes prior to detection of the binding agents. The separation limits the number of unbound binding agents in a compartment, preferably to one, on average. For example, the mean number of unbound binding agents in a compartment is one. For example, separation can be carried out by dilution, specific binding, or separation by physical and/or chemical properties. Compartmentalization can involve locations or physical compartments, or diffusion limited environment. Compartmentalization or separation may comprise any one or more of solid surface binding, dilution or phase separation among the others, or providing diffusion limited or separated compartments. For the compartmentalization, different assays can be envisioned. For instance, an emulsion droplet method may be used to form droplets in an emulsion (e.g. water-in-oil), wherein each droplet represents a separate compartment. As used herein the term “compartment” includes an emulsion droplet, a physical compartment such as a micro-cavity etc., diffusion limited or separated compartments.

The term “droplet” as used herein preferably refers to an isolated portion of a first fluid surrounded by a second fluid. The first fluid comprises preferably a hydrophilic fluid such as water, an aqueous media, or a buffer, and preferably comprises the sample solution or the one or more dilutions thereof to which the bi-component detections system or other reagents are added. The second fluid preferably a hydrophobic fluid, such as hydrocarbons, silicone oils, mineral oils, organic solvent. Emulsions techniques to compartmentalize sample solutions are well known in the art.

In particularly preferred embodiments the compartmentalized bi-component detection method is protein interaction coupling carried out in an emulsion or in a physical compartment. This is a digital assay concept based on the detection of double-labelled (bi-component), individual ternary molecular complexes in emulsion or in physical compartment, which may be identified, for example, by digital PCR (dPCR) or next generation sequencing (NGS). Preferably the method involves droplet digital PCR (ddPCR).

A typical compartmentalized method is the droplet digital PCR (ddPCR) (Quan, Sauzade, & Brouzes, 2018). Droplet Digital PCR (ddPCR) preferably refers to a method for performing digital PCR that is based on water-oil emulsion droplet technology. Droplets are formed in a water-oil emulsion to form partitions (compartments) that separate the template DNA molecules. The droplets serve essentially the same function as individual test tubes or wells in a plate in which the PCR reaction takes place. The oil droplets may be made using a droplet generator that applies a vacuum to each of the wells (see e.g. Pinheiro et al. Analytical Chemistry 84 (2): 1003-11)). Typically a sample may be fractionated into 20,000 or more droplets, and PCR amplification of the template molecules occurs in each individual droplet. Advantageously, ddPCR technology uses reagents and workflows similar to those used for most standard TaqMan probe-based assays. Alternately, dPCR can be carried out in physical nanowells, for example like the Qiagen Qiacuity system.

The massive sample partitioning is a key aspect of the ddPCR technique. The ddPCR technology is digital, in the regard that, the droplets support PCR amplification of the template molecules they contain and generate signals based on individual DNA template molecules.

Following PCR, each droplet is analyzed or read to determine the fraction of PCR-positive droplets in the original sample. These data are then analyzed using Poisson statistics to determine the absolute DNA template concentration in the original sample.

The method may also apply the absolute quantitation principle (Quan et al., 2018) to bi-component measurement methods. The signal of the analyte is based on detection of the individual molecules of the compartmentalized analyte-binding agent complexes (couplexes) and binding agents using Poisson statistics, wherein the analyte-binding agent complexes (couplexes) are determined by the chemical balances based on the concentration of the binding agents and couplexes and their analytical properties, namely dissociation constants.

The creation of tens, thousands or millions (or even higher number) of droplets and their application for the bi-competent detection method means that a single sample measurement can be taken, having the number of droplets as the primary dynamic range of the bi-component method, in an absolute quantitative, highly linear manner. This is in contrast to using a single, inherently non-linear signal obtained by non-compartmentalized measurement methods. The absolute quantitative and statistical features inherent to compartmentalized methods can therefore be used in the detection of analytes using bi-competent methods. These compartmentalization approaches can be combined with the methods such as those described in WO2020260277 in order to provide an extended range of a non-bijective two-segment reference curve of bi-component measurement methods and effectively double the available dynamic range of the measurements.

The methods can be used for multi-analyte measurements as they usually have vast concentration differences. For example, it is possible to measure DNA copy number (low-abundance) and RNA/protein copy number (high abundance) in the same sample using suitable compartmentalized bi-component method such as emulsion coupling (see WO2020260277 and EP 3224360, incorporated herein by reference).

The number of compartments has no upper limits, regarding the signal generation principle, if one applies a digital, linear signal generation method. Due to the extended range of measurement by using the method described in WO2020260277, a doubled signal based dynamic range is available for the measurement, while maintaining sensitivity and precision that are the hallmarks of compartmentalized bi-components methods.

Therefore, the compartmentalized bi-components methods, in combination with the method described in WO2020260277, provide unparalleled precision as the massive sample partitioning enables the reliable measurement of small fold differences in target analytes. This leads to increased signal-to-noise ratio, as the dominating template in a sample does not hamper the detection of rare targets. Compartmentalized bi-components methods provide further low signal drop-out error rates as the inherent high dilution of the methods removes or minimises substances that may interfere with effective signal generation.

The method may utilize parallel reading technologies. Next-generation DNA sequencing is preferred as a bi-component readout technology, particularly those bi-component methods that are suitable for DNA sequencing based readout, which generate unique DNA signals as a part of the detection principles. These methods include, but are not limited to proximity ligation, extension assays and emulsion coupling/protein interaction coupling.

DNA sequencing readout based bi-component measurement methods are preferred, if the bi-component methods is a compartmentalized bi-component method. Under compartmentalized conditions, the generation of unique DNA signals is unbiased, compared to the uncompartmentalized measurements.

The use of the compartmentalized generation of DNA signals means unbiased DNA signals that enable the usage of unique molecular identifiers (UMI) (Parekh, Ziegenhain, Vieth, Enard, & Hellmann, 2017) signal readout.

The bi-component detection method may comprise employing a compartmentalized assay to produce a bi-component/analyte complexes concentration dependent signal, wherein the signal reflects the presence of a pair of analyte-specific binding agents in a single compartment.

Preferably, the compartmentalized assay employs an emulsion droplet or physical compartment method, wherein each droplet or nanowell represents a separate compartment.

The compartmentalized assay may use emulsion coupling/protein interaction coupling.

“Emulsion coupling/protein interaction coupling” as used herein refers to a digital assay concept based on the detection of a pair of binding agents present in an individual ternary molecular complex in an compartment, in particular an emulsion droplet. The pair of binding agents may be identified, for example, by digital PCR (dPCR) using fluorescently tagged PCR products or next generation sequencing (NGS).

Advantageously, such an approach allows for an absolute quantification of analyte concentration as well as a parallel determination of the concentration of multiple analytes in a robust and efficient manner.

The bi-component detection method may comprise employing an absolute molecular count based analytical method. Such an absolute molecular count based analytical method preferably refers to applying digital detection methods, such as a digital PCR or next-generation sequencing.

The bi-component detection method may comprise employing a droplet digital PCR assay. For example, the bi-component detection method may comprise using analyte-specific binding agents associated with unique amplifiable nucleic acid labels and employ a compartmentalized assay, wherein a nucleic acid amplification is performed for each compartment using fluorescently tagged amplification products.

Preferably, the nucleic acid amplification is a PCR and the fluorescently tagged amplification products are fluorescently tagged PCR products.

The analyte-specific binding agents (such as an antibody pair) may preferably be labelled by unique PCR amplifiable DNA labels, that is the pair of analyte-specific binding agents, e.g. two antibodies, may preferably be labeled with a single stranded DNA that uniquely identifies the binding component (e.g. antibody). The labelled binding agents (e.g. antibodies) are added to the sample or the one or more dilutions thereof in order to allow for bi-component/antibody complex formation.

Before an emulsification of the samples, the reaction is highly diluted (for example upto 100,000 times) and PCR reagents are added to achieve near single-molecule separation and PCR amplification per compartment. dPCR may be carried out using the standard dPCR protocol. The reaction is preferably highly diluted, e.g. by a dilution factor of more than 1 000, preferably more than 10 000, 100 000 to achieve single-couplex separation upon a compartmentalization, e.g. by emulsification into droplets. Each ternary complex or couplex is nearly isolated in an emulsion droplet prior to detecting the presence of at least two pairs of binding agents.

For the nucleic acid amplification, fluorescently tagged amplification products are used that recognize the binding component (e.g. antibody) specific unique amplifiable nucleic acid labels. For instance, fluorescently tagged PCR products, e.g. using FAM- or VIC-labelled real-time PCR probes may be used that are complementary to the single stranded DNA that uniquely identifies the binding component.

The nucleic acid amplification is performed in each of the compartments, e.g. emulsion droplets and a signal readout may be performed by detecting the fluorescence signal of the compartments, e.g. the ‘colour’ of the droplets.

The evaluation of the reaction may be based on the partitioning of the labels in a dPCR reaction using fluorescently tagged PCR products (e.g. using FAM- or VIC-labelled real-time PCR probes). According to the dPCR, standard evaluation the cluster of droplets may be determined according to the fluorescent signals of the droplets. Herein, the number of labelled binding agents (e.g. antibodies) is determined in each reaction (counting all label-positive droplets for a given label, and using the same definition of cluster of droplets for all reactions).

Additionally, the number of the double-colored (having two different binding component labels) is also determined. Without ternary complexes, the partitioning of the labelled antibodies follows Poisson distribution, and results in a calculable number of double-colored droplets (having two binding agents in one compartment based upon pure chance). In the case of ternary complexes present in the reaction, the number of the detected double-colored droplets (having additional ternary complexes) is larger than would be expected by Poisson distribution. Based upon such an analysis the number of ternary complexes can be thus calculated. Advantageously, this allows for an absolute quantitation of the ternary analyte complexes formed.

On the basis of these measurements the number of couplexes can be calculated based on known methods (see e.g. EP 3224360 or Karakus et al., 2019). This results in an absolute (the count of molecules) quantitation of the ternary analyte couplexes. In addition, the number of couplexes containing one or more pairs of binding agents can be calculated. This provides details of which epitopes are present and bound by the binding agents within the couplex. The knowledge of which pairs of binding agents are present in the couplex allows the concentration of a target present to be calculated. Utilizing the information from two or more pairs, the characteristics of the analyte in the couplex, and so a sample can also be determined. For example, if one of the binding agents is capable of binding to a modified version of an analyte the presence of the modified/unmodified form in a sample can be verified and quantified. Similarly, if the sample contains interacting molecules, by using four or more binding agents the presence and quantity of each molecule, as well as the interacting molecules can be determined. If two binding agents bind to each molecule, one pair can be used to determine the quantity of each molecule. The signal generated by a pair of binding agents, each of which binds to a different member of the interacting molecules can be used to detect and quantify the presence of the interacting molecules.

Preferably, dPCR is employed and according to a standard evaluation of dPCR, the number of droplets may be determined according to the fluorescent signals of the droplets. Herein, the number of labelled binding agents (e.g. antibodies) is determined in each reaction (counting all label positive droplets for a given label, and using the same definition of cluster of droplets for all reactions). Additionally, the number of the double-colored or multi-colored (having two or more different pairs of labelled binding agents) is also determined.

Multiple analytes may be determined in parallel. For example, the bi-component detection method comprises using multiple analyte-specific binding agents comprising nucleic acid barcodes and employs a compartmentalized assay, wherein a nucleic acid amplification is performed for each compartment producing linked nucleic acid barcodes. The compartments are reunited in a common pool and a parallel nucleic acid sequencing technique is used to produce the bi-component/analyte complexes concentration dependent signal.

The binding agents comprising nucleic acid barcodes may be antibodies labelled with unique PCR amplifiable DNA labels, comprising a unique label for the type of antibody used and also a label for the individual molecule (unique molecular identifier-UMI) (see also Parekh et al., 2017). The thus labelled antibodies are added to the sample in order to allow for bi-component/antibody complex formation.

Performing the nucleic acid amplification for each compartment to produce linked nucleic acid barcodes may be achieved by highly diluting the sample before the nucleic acid amplification, e.g. with a dilution factor of more than 1 000, preferably more than 10 000, more preferably more than 100 000. In preferred embodiments, the nucleic acid amplification is PCR. PCR reagents may be added to achieve near single-molecule separation and nucleic acid amplification per compartment. To this end digital PCR standard protocols may be particularly suited.

Afterwards the compartments, e.g. emulsion droplets, may be recombined in a common pool and a parallel nucleic acid sequencing technique can be used to assess antibody specific dimerized UMI labels. Herein, the number of labelled antibodies can be determined in each reaction by counting all unique UMI labels for a given antibody (counting restricted to a given antibody—e.g. in a given label context). A possible multiple labeling of the same antibody can be eliminated using their preferentially dimerized sequences, since multiple labels per antibody exhibit a double UMI label dimers with a given antibody specific label context, as they are co-localizing in the same droplet.

The ternary bi-component/antibody complexes can be counted on the basis of their dimerized double UMI labeled PCR products generated from two different antibody specific labels, called (heterodimers) with a correction according to the multiple antibody labels.

A preferred parallel nucleic acid sequencing technique used herein is a next generation sequencing technique.

“Next generation sequencing (NGS)” as used herein shall encompass recently developed technologies for the sequencing of nucleic acids that typically allow much higher throughput than the traditional Sanger approach (see Schuster, Next-generation sequencing transforms today's biology, Nature Methods 5:16-18 (2008); Metzker, Sequencing technologies the next generation. Nat Rev Genet. 2010 January; 1 1 (1): 31-46. These platforms can allow sequencing of clonally expanded or non-amplified single molecules of nucleic acid fragments. Certain platforms involve, for example, sequencing by ligation of dye-modified probes (including cyclic ligation and cleavage), pyrosequencing, and single-molecule sequencing. Nucleotide sequence species, amplification nucleic acid species and detectable products generated there from can be analyzed by such sequence analysis platforms. Next-generation sequencing can be used in the methods of the invention, e.g. to quantify unique PCR amplifiable DNA labels in order to assess the formation of bicomponent/analyte complexes as described below.

The details of emulsion coupling/protein interaction coupling as a preferred compartmentalized bi-component detection method are described in EP 3224360, which is incorporated by reference herein. For a dPCR detection in an emulsion coupling assay the analyte-specific binding agents (such as an antibody pair) are labelled by unique PCR amplifiable DNA labels and the thus labeled binding agents are added to the sample (or dilutions thereof see e.g. Example 3).

For a next generation sequencing detection in an emulsion coupling/protein interaction coupling assay the binding agents may preferably be labelled by unique PCR amplifiable DNA labels, comprising a specific label for binding component (e.g. antibody) and preferably also an individual label for the molecule, i.e. a unique molecular barcode or unique molecular identifier-UMI (see Parekh et al., 2017). The labeled binding agents may be added to the samples or dilutions thereof (see Example 3).

After binding of the binding agents, e.g. the antibodies, and before the emulsification of samples, the reaction may be highly diluted e.g. with a dilution factor of more than 1 000, preferably more than 10 000, more preferably more than 100 000; and PCR reagents may be added to achieve near single-molecule separation. PCR amplification can be performed per compartment. dPCR may be carried out using dPCR protocol.

The evaluation of the reaction may be based on the NGS reading of the binding component, e.g. antibody, specific dimerized UMI labels generated according to the standard protocol of emulsion coupling. The number of labelled binding agents, e.g. antibodies, may be determined in each reaction by counting all unique UMI labels for a given binding component, e.g. antibody (counting restricted to a given binding component). Possible multiple labelling of the same binding component, e.g. antibody, can be eliminated using their preferentially dimerized sequences (multiple labels per binding component will always result in double UMI label dimers with a given antibody specific label context, as they co-localizing in the same droplet).

Ternary antibody/bi-component complexes are counted based on their dimerized double UMI labeled PCR products (in the context of two different binding component (e.g. antibody) specific labels called heterodimers). A correction for multiple labeling of binding agents (e.g. antibodies) may be used following the concept described above, which takes into account double UMI label dimers with a given component (e.g. antibody) specific label.

A further evaluation of the samples can be carried as in case of fluorescently tagged PCR (digital PCR). Briefly without ternary complexes, the partitioning of the labelled antibodies follows Poisson distribution, and results in a calculable number of ternary complexes (based on the detection of heterodimers) in droplets (having only two antibodies by chance). In the case of ternary complexes present in the reaction, the number of the detected heterodimers is larger than would be expected by pure Poisson distribution. On the basis of this measurements the number of complexes can be calculated using known methods (see EP 3224360, Karakus et al., 2019)). This results in the absolute (the count of molecules) quantitation of the ternary complexes.

The dilutions of the samples can be measured in the same sequencing reaction using samples specific DNA barcodes (e.g. barcoded primers) and as antibodies have distinguishable component (e.g. antibody) specific labels many measurements (using different antibody pairs against different antigens) can be carried out in parallel.

In embodiments of emulsion coupling/protein interaction coupling the binding agents may be provided as a library of binding agents suitable for the detection of multiple analytes. In these embodiments, each member of the binding component library may be associated with a unique nucleotide sequence, which can be used to identify the binding component.

In emulsion coupling/protein interaction coupling, the presence of the binding agents in the complex may be detected by the presence of the nucleic acid sequence within the linked sequence generated in the method. The nucleotide sequence may be attached as a label to the binding component, be part of the binding component itself e.g. aptamer, or be present within the binding component e.g. nucleic acid within a phage. For example, each member of the library can be labelled with a unique nucleotide sequence that is a nucleotide sequence is attached to the binding agents.

Methods of attaching nucleotides to binding agents such as antibody or compounds are known in the art. Alternatively, if the binding component library is a phage display library the unique nucleotide sequence can be the sequence that encodes one or more CDR regions or the displayed binding domain. For example, a display library can be generated by inserting sequences encoding the amino acid sequence to be displayed into a phage at a known location. Universal primers that will amplify the inserted sequences can then be used and thus identify the binding sequence. Alternatively, if the binding component may be an aptamer and the aptamer itself can be the unique nucleotide sequence.

The method of the present invention may utilize a computer programme product, such as a software product.

The software may be configured for execution on common computing devices and is configured for carrying out one or more of steps of the method described herein.

The computer program may be configured for comparing signals detected in the sample and in one or more dilutions with the analyte concentration reference curve in order to determine the concentration of the analyte in the sample or further preferred embodiments of the computational steps as disclosed herein.

The computer program may be configured for performing the computational steps of determining the dissociations constants kd1 and kd2 of said two analyte-specific binding component using said signal detected in the sample and in the one or more dilutions as a constraining input for a mathematical fit for said dissociation constant relationship at given binding agents and analyte concentrations.

The invention will now be described in the examples below which refer to the following figures:

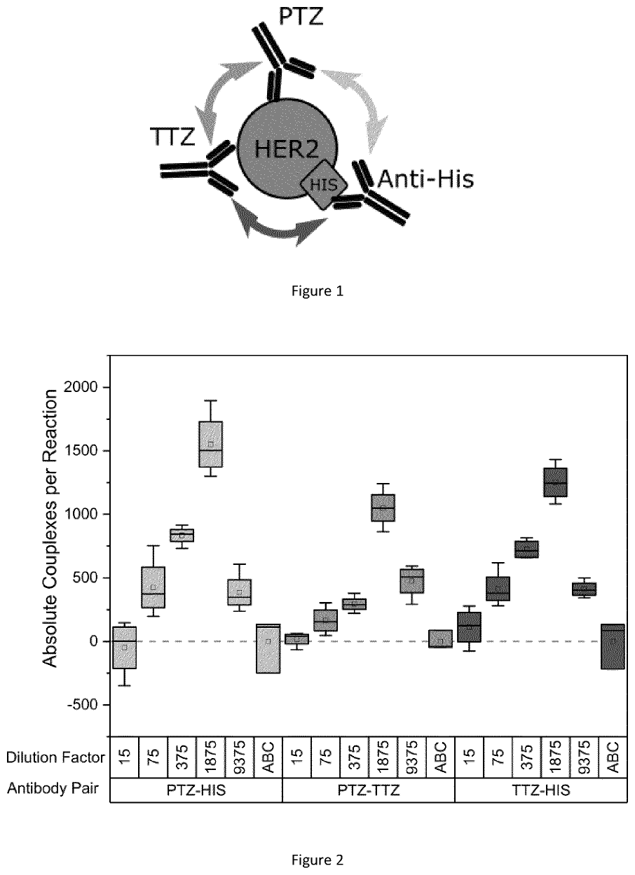

FIG. 1 shows ‘Three-way absolute homogeneous internal multiplexing readout’ rHER2 protein interaction coupling assay using trastuzumab, pertuzumab and anti-HIS.

FIG. 2 shows ‘Three-way absolute homogeneous internal multiplexing readout’ calibration curve—CLC—of rHER2 using trastuzumab, pertuzumab and anti-HIS (see details in the text). ABC correction is applied. Note the zero background in case of highly concentrated samples (dilution Factor-15), these samples are not significantly different from zero readout of couplexes, as expected. Boxplots are with median (line), mean (dot), interquartile range (IQR) between third quartile and 1.5×IQR whiskers (four parallels, except for ABC which is three parallels). Couplexes are per reaction of a well of QIAcuity Nanoplate 26k 24-well plate. Samples dilutions and antibody pairs are indicated, see text for details.

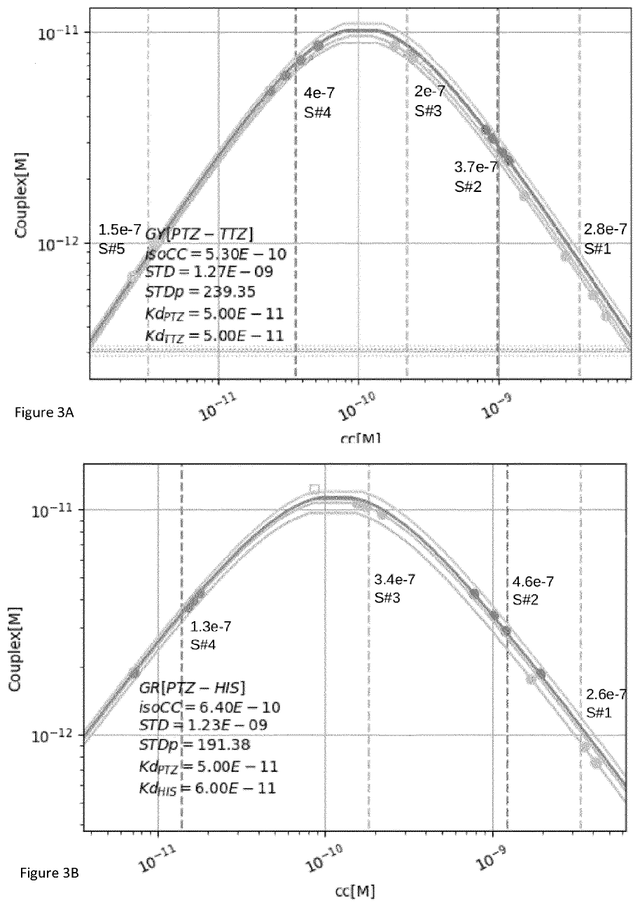

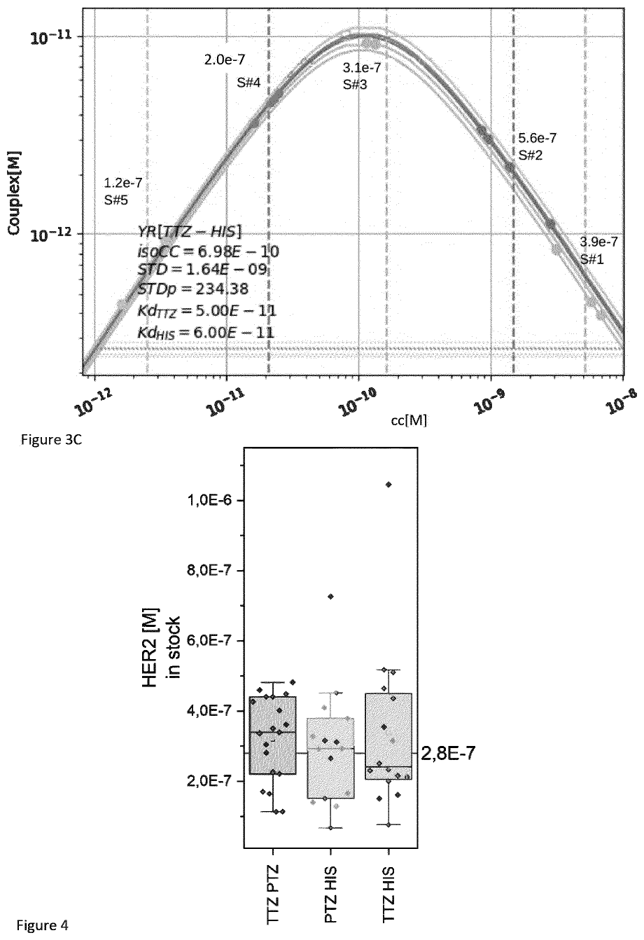

FIG. 3 shows ‘Three-way absolute homogeneous internal multiplexing readout’ of calibration curves of rHER2 using trastuzumab, pertuzumab and anti-HIS (see details in the text). ABC correction is applied. Samples #1-#5 are indicated with the dilution compensated antigen concentrations. The legend indicates the color pair GY—green-yellow, GR—green-red, YR—yellow-red, antibodies TTZ—trastuzumab, PTZ—pertuzumab, HIS—anti-HIS antibody. Y axis is molar couplex concentration, X axis is recombinant HER2 concentration, both are logarithmic.

FIG. 4 shows. ‘Three-way absolute homogeneous internal multiplexing readout’ of antigen concentrations. The legend indicates antibodies TTZ—trastuzumab, PTZ—pertuzumab, HIS—anti-HIS antibody. Y axis is absolute molar concentration of recombinant HER2. Background line at 2,8E⁻⁷ is the reference concentration of the stock rHER2 measured.

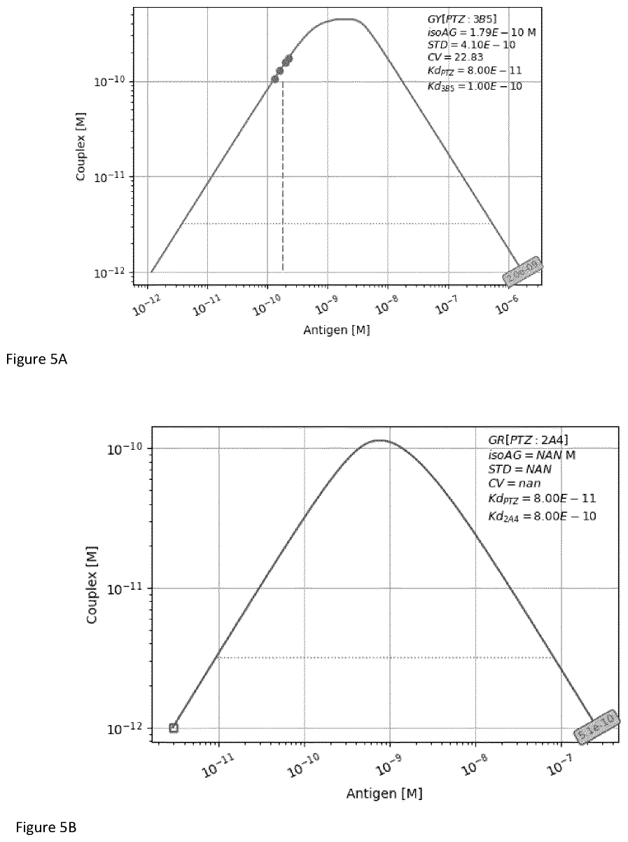

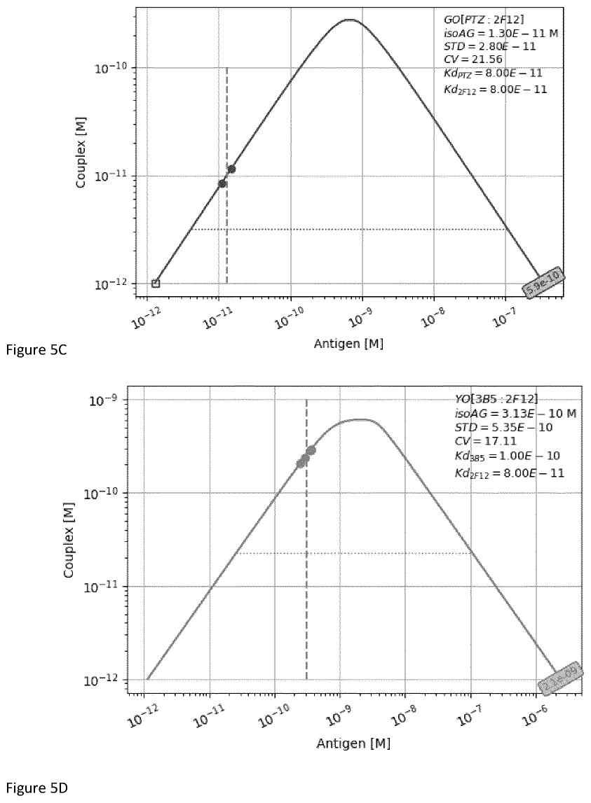

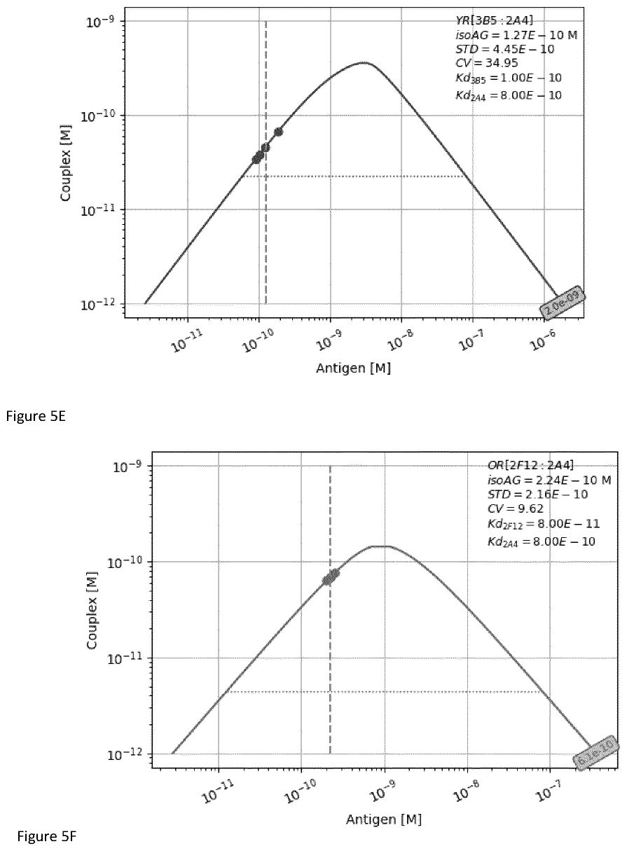

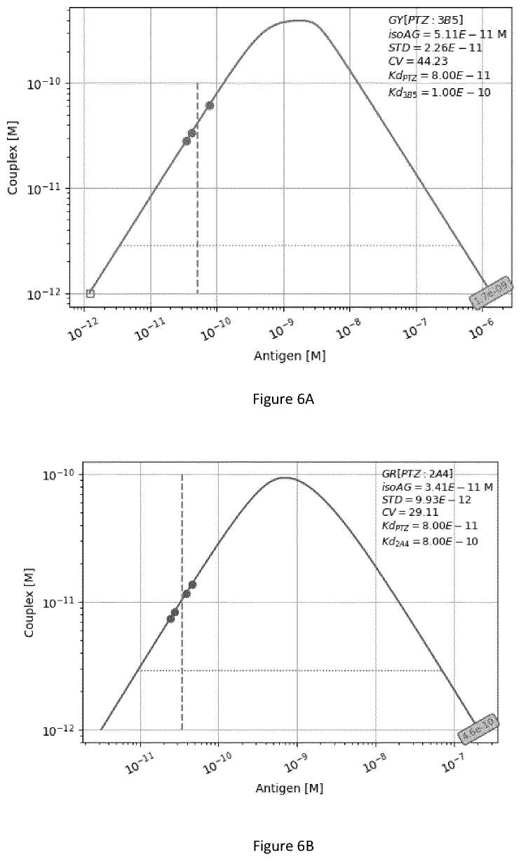

FIG. 5 shows ‘Four-way absolute homogeneous internal multiplexing readout’ of calibration curves of HER2-HER3 of 2000 of BT474 cells (1:10 dilution), using pertuzumab (PTZ) was labeled with the P8 label (green—G), ErbB2 3B5 was associated with the BL label (yellow—Y), ErbB3 2F12 was labeled with the N6 label (orange—O) and ERBB3 2A4 was associated with the O7 label (red—R), see details in the text. ABC correction is applied. Samples #1-#5 are indicted with the dilution compensated antigen concentrations. The legend indicates the color pair GY—green-yellow, GR—green-red, YR—yellow-red. Y axis is molar couplex concentration, X axis is analyte (HER2m HER2:HER3 interaction and HER3, depending the antibody pairs measured) concentration, both are logarithmic. The horizontal dotted line is the calculated LOD (limit of detection). Legend, in first line the color-pair and the antibody pair are indicated, isoAG (isomolar concentration of antigen) is the mean concentration of analyte indicated by the red dotted line, STD is standard deviation of the antigen concentration and CV is the coefficient of variation in percentage. Kds of the antibodies are also given, determined according to WO2020260277A1, see above.

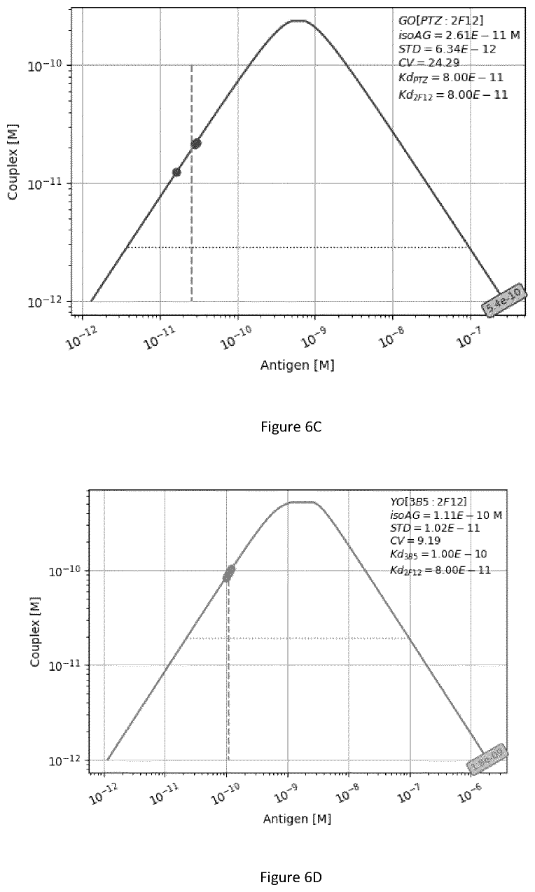

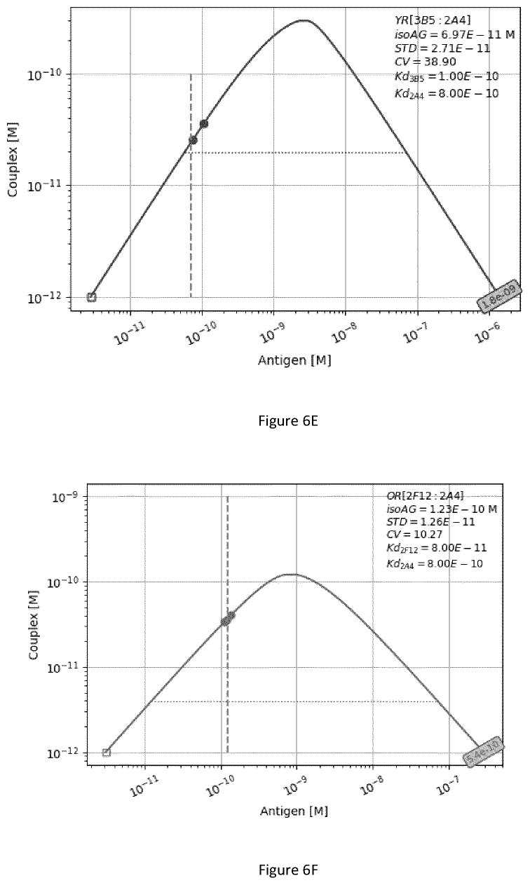

FIG. 6 shows ‘Four-way absolute homogeneous internal multiplexing readout’ of calibration curves of HER2-HER3 of 20,000 of MCF7 cells (no dilution), using pertuzumab (PTZ) was labeled with the P8 label (green—G), ErbB2 3B5 was associated with the BL label (yellow—Y), ErbB3 2F12 was labeled with the N6 label (orange—O) and ERBB3 2A4 was associated with the O7 label (red—R), see details in the text. ABC correction is applied. Samples #1-#5 are indicted with the dilution compensated antigen concentrations. The legend indicates the color pair GY—green-yellow, GR—green-red, YR—yellow-red. Y axis is molar couplex concentration, X axis is analyte (HER2, HER2:HER3 interaction and HER3, depending the antibody pairs measured) concentration, both are logarithmic. The horizontal dotted line is the calculated LOD (limit of detection). Legend, in first line the color-pair and the antibody pair are indicated, isoAG (isomolar concentration of antigen) is the mean concentration of analyte indicated by the red dotted line, STD is standard deviation of the antigen concentration and CV is the coefficient of variation in percentage. Kds of the antibodies are also given, determined according to WO2020260277A1, see above.

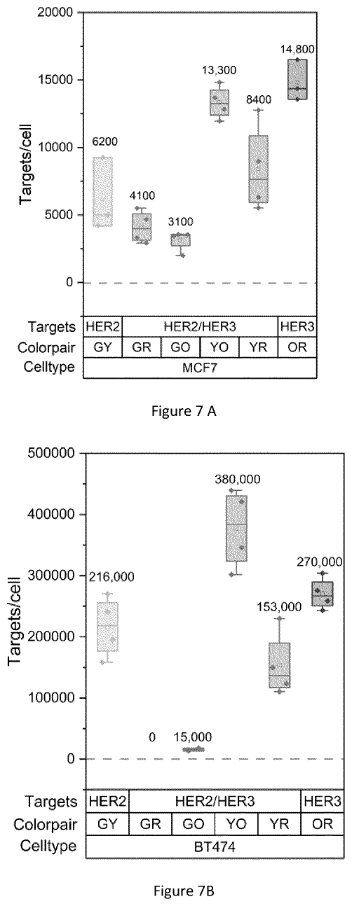

FIG. 7 shows ‘Four-way absolute homogeneous internal multiplexing readout’ data of HER2, HER3 and their interaction using BT474 and MCF7 cells were measured and absolute quantified as above. Absolute copies of analytes are given per cell, per antibody pairs, where GY measures the HER2 protein copies per cells, GR, GO, YO and YR measures the HER2:HER3 protein interaction copies per cells and finally OR measures the HER3 protein copies per cells. Please note that PTZ (pertuzumab-green—G) has very limited or no access to cross-linked HER2:HER3 complex and other epitope steric hindering also occurs.

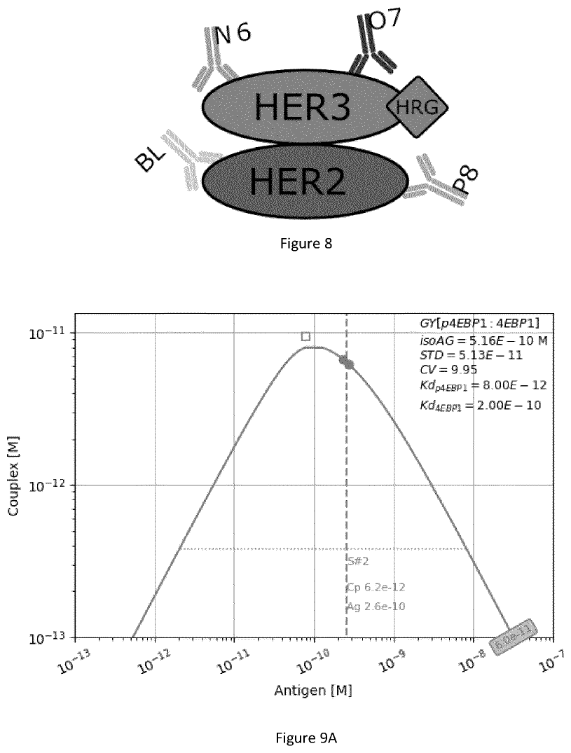

FIG. 8 shows Absolute quantitative ‘Four-way absolute homogeneous internal multiplexing readout’ data of HER2, HER3 and their interaction using BT474.

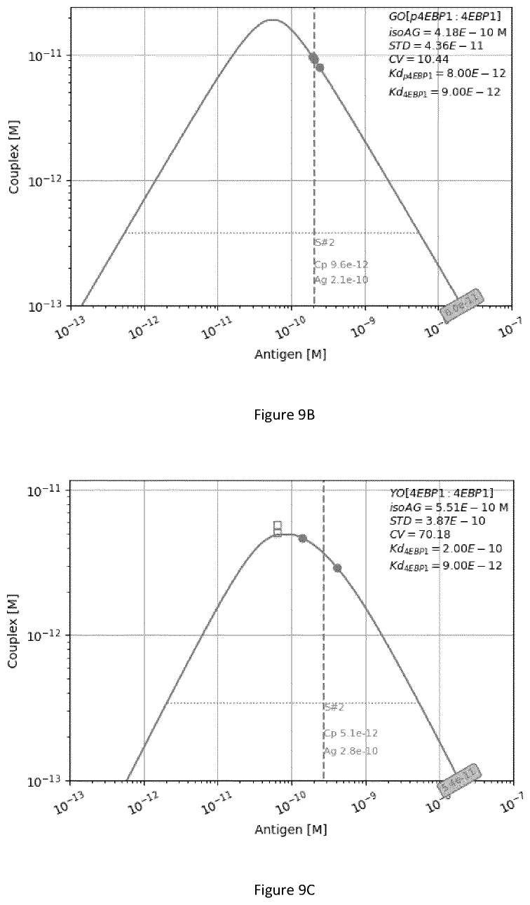

FIG. 9 shows ‘Four-way absolute homogeneous internal multiplexing readout’ of calibration curves of 4EBP1-p4EBP1 of 10,000 of U937 cells (two times dilution), using phospho-4EBP1 (Thr37, Thr46) 4EB1T37T46-A5 was labeled with the P8 label, 4EBP1 554R16 was labeled with the BL label, EIF4EBP1 clone 4F3-H2 was labeled with the N6 label and 6*His, His-Tag as a unspecific antibody (UAB) was labeled with the O7 label. Samples S #2 indicates the antigen concentration. The legend indicates the color pair GY—green-yellow, GO—green-orange, YO—yellow-orange. The antibody pairs with the red antibody (UAB) are all zero and not depicted. Y axis is molar couplex concentration, X axis is analyte (4EBP1 or p4EBP1, depending the antibody pairs measured) concentration, both axises are logarithmic. The horizontal dotted line is the calculated LOD (limit of detection). Legend, in first line the color-pair and the antibody pair are indicated, isoAG (isomolar concentration of antigen) is the mean concentration of analyte indicated by the red dotted line, STD is standard deviation of the antigen concentration and CV is the coefficient of variation in percentage. Cp—concentration of couplex, Ag—concentration of antigen. Kds of the antibodies are also given, determined according to WO2020260277A1, see above.

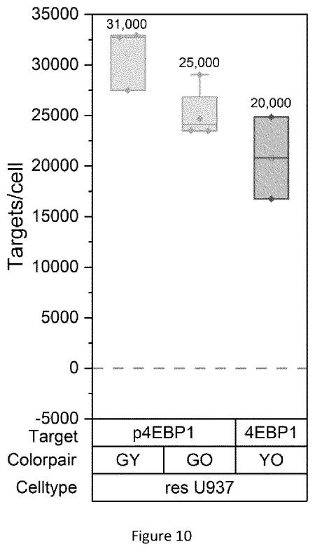

FIG. 10 shows Absolute quantitative ‘three-way absolute homogeneous internal multiplexing readout’ data of 4EBP1-p4EBP1 using U937 cells were measured and absolute quantified as above. Absolute copies of analytes are given per cell, per antibody pairs, where GY and GO measure the p4EBP1 protein copies per cells, YO measures the 4EBP1 protein copies per cells and finally all R pairs measures the unspecific copies per cells (not depicted, all are zero).

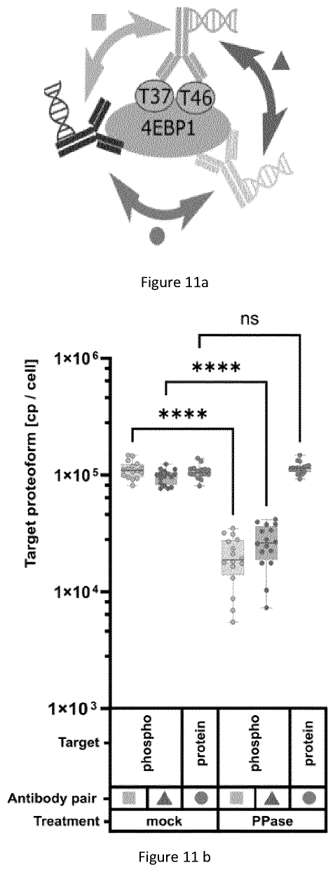

FIG. 11a shows quantitative ‘three-way absolute homogeneous internal multiplexing readout’ data of 4EBP1-p4EBP1. Antibody pairs are indicated: circle—Anti-4EBP1 4F3-H2 and Anti-4EBP1 2C3F3, measuring 4EEBP1 protein amount; square—Anti-4EBP1 4F3-H2 with Anti-Phospho 4EBP1 MA5-36935 measuring the phospho-4EBP1, and triangle—Anti-4EBP1 2C3F3 with Anti-Phospho 4EBP1 MA5-36935 also measuring the phospho-4EBP1 FIG. 11b shows quantitative ‘three-way absolute homogeneous internal multiplexing readout’ data of 4EBP1-p4EBP1 using U937 cells. Bulk PICO assay—20,000 cells in a 4 μL volume, measuring absolute quantitative amounts (AQ) of 4EBP1 and p-4EBP1 in U937 bulk lysate at 5⁻¹⁰M of antibody concentration, treated with λ-phosphatase (PPase or left untreated (mock). Untreated samples showed comparable 4EBP1 and p-4EBP1 AQ levels, while λ-phosphatase treatment significantly reduced the p-4EBP1 signal, as expected.

### EXAMPLES

The protein interaction coupling workflow consists of three main parts—the immune reaction part (antibody binding) and the dPCR part (digital PCR) followed by the evaluation of the protein interaction coupling results.

The immune reaction part is a simple antigen-antibody equilibrium binding reaction using a mixture of DNA-amplicon-labeled antibodies. The typical the setup has a volume of reaction of a few microliters, consisting of the sample and the antibody mix (ABX), which is typically incubated overnight to achieve equilibrium binding. The sample can be almost any type of soluble protein material, as during the dPCR phase-due to the applied high dilution-almost no chemical interference is expected, assuming the undisturbed binding of the antibodies. Since protein interaction coupling counts molecules (both antibodies and couplexes), it is important to achieve molecular dispersion of the samples, so the lysis conditions are optimised to achieve molecular dispersion even for membrane proteins. The assay has no washing steps (e.g. homogeneous assay) ensuring unbiased concentration readings of both, couplexes and antibodies.

After incubation, due to the large number of formed couplexes and antibodies in the assay, the reaction must be diluted to achieve an antibody count that is less than the number of dPCR partitions, the measurable range of a dPCR reaction. The dilution achieves on average one couplex per partition/compartment. The diluted sample is combined with the dPCR master mix (DPMX) and the dPCR is carried out.

### Example 1. General Workflow of Protein Interaction Coupling

### General Considerations Regarding the Assay Conditions

For dPCR the contamination is less critical than for ordinary PCR reactions, as the compartments contain the amplification. NTC (non-template control) is carried out to detect the contamination. The experiments carried out at two physically separated workstations: (1) pre-dPCR bench for steps of reagent setup, cell preparation, (2) dPCR bench for steps with high oligonucleotide contamination potential. The assay is sensitive to variations in pipetting volumes, so calibrated pipettes are used. Vortexing means 10 sec vortexing at high speed, as low-efficiency vortexing can introduce large standard deviations. Spin centrifugation means low speed (1000 g for 30 seconds) in a minifuge.

### Devices

QiaCuity One, 5plex Device (Qiagen)
Cell counter Countess II (Invitrogen, #A27977)
Centrifuge (up to 21,000 g and with cooling function)
Plate centrifuge Megafuge 8 (Thermo Fisher Scientific)
Ultrasonic bath Sonorex Super (Bandelin) (size for a 96-well microplate)
Minifuge (Biozyme, #55C1008-B-E)
Electronic Multichannel pipette (INTEGRA, #4722), 12-channel, 5-12.5 μl
Multichannel pipette, 8-channel, 10-100 μl
Multichannel pipette, 8-channel, 30-300 μl
Regular 1-channel pipettes (1-1000 μl)

### Materials

complete Protease™ Inhibitor Cocktail (Roche Cat #4693159001)
QIAShredder
Trypan blue
Recommended: DMEM (DMEM/F12 10% FBS)
Falcon tubes (15 ml and 50 ml)
Phosphate-Buffered Solution (PBS), should not contain calcium or magnesium ions (Thermo Fisher Scientific; Ser. No. 12/037,539)
Microplate, 96 well, Polypropylen, U- or V-bottom
Eppendorf-style reaction tubes

### 5% BSA Stock

20 mg BSA
PBS to 400 μl
Stable for 3 days at 4° C.

### EDTA-Free Protease Inhibitor Cocktail (PIC), 25× Stock

1 tablet of complete Protease™ Inhibitor Cocktail (Roche Cat #4693159001)
PBS to 2 ml
Stable for 12 weeks at −20° C.

### PIC-PBS, Working 1×

1 tablets into 50 ml Falcon tube of PBS
Stable for 3 days at 4° C.

### Cell Lysis Stock (CLS), 10×

20 mM Tris-HCl (pH 7.5)
150 mM NaCl
1 mM Na₂EDTA
1 mM EGTA
1% Triton
2.5 mM sodium pyrophosphate
1 mM beta-glycerophosphate
1 mM Na3VO4
1 μg/ml leupeptin

### Cell Lysis Buffer 2×Stock (LBT)

200 μl 0.5% Tween-PBS (Tween-20 Sigma, Cat #P9416-50ML)
400 μl 80 mM Chapso (CHAPSO Roth, Cat #NH_{73.1})
80 μl PIC (25×)
200 μl Cell Lysis Stock (10×)
PBS to 1000 μl
   Stable for 3 days at 4° C.

### Cell Lysis Buffer (LBTW) 1×Working

750 μl Cell Lysis Buffer 2×stock
PBS to 1500 μl
Stable for 3 days at 4° C.

### BS3 Cross-Linker Stock Solution (BS3S) 100 mM 20× Stock

BS3 is supplied at 2 mg/vial, (Thermo Fisher Scientific; #A39266), the stock is prepared by adding 37.8 μl DMSO (water-free) to the vial. Stable at −20° C. for several months.

### BS3 Cross-Linker Working Solution (BS3W) 5 mM 1× Working

6 μl BS3 stock+114 μl PBS, freshly prepared

### ABC Buffer 1× Working

250 μl Cell Lysis Buffer 2× stock
100 μl 5% BSA stock
PBS to 500 μl

### Cell Cultivation

U937 cells were cultivated in Nunc™ EasYFlask™ Nunclon™ Delta Surface (Thermo Scientific) using the cell culture medium RPMI 1640 Medium, GlutaMAX™ Supplement (1×) (Cat #61870010, Thermo Fisher Scientific)+10% FBS (Gibco) and 1% Pen-Strep (Gibco) at 37° C./5% CO2.

### Preparation of the Cells and Cross-Linking

The EDTA-free Protease Inhibitor Cocktail (PIC) (25× stock), and additional PIC-PBS working 1× and BS3 Cross-linker Stock Solution (BS3S) 100 mM 20× stock solutions were prepared. The cells were washed with 1 ml PBS two times (400 g, 5 min) and the cells were pelleted by centrifugation (400 g, 5 min) and removed. The crosslinking of the cells was by adding 100 μl of 5 mM BS3W, mix by gently pipetting up and down and incubating at RT for 30 min. The BS3W was prepared shortly before use. LBT and LBTW were also prepared in the meantime. Short-arm cross-linking used here acts only in short-distances between primary amines of proteins (homobifunctional crosslinker), and results in mild cross-linking of proteins prominently with interactions. The BS3 is a non-membrane permeable crosslinker so it exerts its action only on the extracellular protein domains. Cross-linking is not required theoretically to detect proteins or posttranslational modifications with protein interaction coupling assay, however it is still recommended as it acts as enzyme inactivator regarding both proteases and nucleases. However, B3 is not absolutely necessary (even can limit it, see below), and as it has only a surface cross-linking effect (it is membrane non-permeable) it is not suitable for intracellular cross-linking. The BS3 effect has been proved, by using pertuzumab (PTZ) as a probe of the cross-linked HER2:HER3, as PTZ unable to bind the cross-linked complex, and as consequence, the PTZ-TTZ (trastuzumab-TTZ) HER2 assay measures less HER2 after BS3 cross-linking. Also noted, that the detection of protein interaction is not fully dependent on stabilizing effect of cross-linking. As the conditions applied are compatible with colP conditions, similar stability of complexes as it is seen in colP environment are expected. However, as colP is just semi-quantitative and less sensitive quantitative and even qualitative differences between colP and protein interaction coupling results are expected and cross-linking is generally recommended for measuring protein interactions. The cells were washed three times with 1 ml fresh PIC-PBS (400 g, 5 min) the supernatant was discarded each time, on ice, at 4° C. and with ice-cold buffers in the subsequent steps. The cells were counted using Countess II cell counter and aliquots of 1 million cells were made at a concentration of 1 million cells per 100 μl (=1×10⁴ cells/μl).

### Cell Lysis

The cells were centrifuged at 400 g for 5 min and the supernatant was carefully discarded, as the number of the cells in the assay is defined at this step. By adding 100 μl of LBTW to the cell pellet, the cells were resuspended and vortexed briefly. The lysis of cells was carried out for 3 h at 4° C. LBTW contains both protease and phosphatase inhibitors to protect the targets, however it also secures the integrity of labelled antibodies against proteases and nucleases during the overnight incubation step. The lysate was sonicated for 5 min at full power in an ultrasonic bath, at room temperature, as maintaining the temperature around 4° C. is not critical in this step, however some proteins might need extra precautions. The lysate was transferred into a QIAshredder and centrifuged for 2 min at full speed (~20,000 g), the flow-through was transferred to a fresh tube. The lysate is ready to use. Molecular dispersion of the sample is an important prerequisite of the protein interaction coupling assay, the protocol above works well with most membrane proteins, however some proteins and interacting protein complexes can be denatured by freezing.

### The Properties of the Protein Interaction Coupling Reaction

Protein interaction coupling has a bell-shaped calibration curve, a relationship between the measured concentration of couplexes and the varied antigen concentrations. The standard curve is constructed at a predefined concentration of antibodies. The curve is defined by measuring the couplex concentrations by combining the antibody with varied dilutions of the target containing sample. Anticipated results include the limit of detection (LOD) and dynamic range of detection (DRD). An unknown sample can be relatively quantified by comparing the couplex concentration of the unknown sample to the calibration curve. As the calibration curve is bell-shaped meaning that one couplex concentration corresponds to two different cell equivalent values it is recommended measuring at least two samples with a defined dilution step apart and fit the couplex concentrations to the curve, a preparation for a sample with unknown amount of target.

Limited by the necessary dilution before the dPCR step of the protein interaction coupling assay, the lowest applicable antibody concentration is approx. 1×10⁻¹² M (QIAcuity Nanoplate 26k 24-well, Cat No./ID: 250001). The upper limit is the maximum achievable labeled antibody stock concentration (limited by the antibody stock concentration itself), however the protein interaction coupling assay is more sensitive at low antibody concentrations, so for this practical and also economical reasons a starting antibody concentration of 8×10⁻¹¹ M (ABX) is recommended.

### Preparation of the Antibody Mix (ABX) for the Calibration Curve Experiments

Two carefully chosen antibodies were labelled using a method of choice (see below) using the labels below. The ABC buffer, and the ABX in ABC Buffer were prepared according to the recommendations for the concentration of the antibodies. The strategy to titrate the concentration of antibodies against a typical sample was carried out as a preliminary experiment to set up a protein interaction coupling assay with unknown amount of target and not yet characterized antibodies (called isomolar titration-IMT). The protein interaction coupling assay has the highest sensitivity at a given concentration of antibodies, so using a higher or lower concentration of antibodies both can result in lower sensitivity. However a typical antibody concentration is 8×10⁻¹¹ M and can be used as a starting concentration without prior titration.

BL label
(SEQ. ID No: 1)
(5′-TTTTTGGTGACGATCCCGCAAAA-TCCAATGATGAGC
ACTTTT---TGCAAGCCTCAGCGACC-3′)
P8 label
(SEQ. ID No: 2)
(5′-TTTTTGGTGACGATCCCGCAAAA-GCGGCCTTTAACTC
CC------TGCAAGCCTCAGCGACC-3′)
O7 label
(SEQ. ID No: 3)
(5′-TTTTTGGTGACGATCCCGCAAAA-TCCCTCCTAGTTC
CCC------TGCAAGCCTCAGCGACC-3′)
N6 label
(SEQ. ID No: 4)
(5′-TTTTTGGTGACGATCCCGCAAAA-TCACCTACCGGC
CTCC------TGCAAGCCTCAGCGACC-3′)

### Binding Reaction

2 μl of cell lysate with 2 μl ABX were combined in a V-bottom 96-well PCR microplate with replicas of four. For ABC control, 2 μl ABC Buffer with 2 μl of ABX were combined. For NTC (non-template control) 4 μl of LBTW to the dedicated well was added. The plate was sealed with an adhesive foil and sonicated at full-power (100%) for 1 min floating in an ultrasonic bath. The plate was centrifuged briefly to collect the liquid at the bottom, and incubated at 4° C. overnight.

Dilution of the Samples Before dPCR

For one QIAcuity Nanoplate 26k 24-well (Qiagen, Cat #250001) a total of 1050 μl mastermix are prepared. Therefore, 262.5 μl of QIAcuity Probe Mix (Qiagen, Cat #250101), 33.6 μl of Primer Mix containing 25 UM forward primer and 25 UM reverse primer, 42 μl of 10 μM MGB probes mixed.

Forward primer
(SEQ. ID No: 5)
(5′-GGTGACGATCCCGCAAAA-3′)
Reverse primer
(SEQ. ID No: 6)
(5′-GGTCGCTGAGGCTTGCA-3′)
MGB probe BL
(SEQ. ID No: 7)
(Eurofins; 5′-HEX-CAATGATGAGCACTTTT-MGBEQ-3′)
MGB probe P8
(SEQ. ID No: 8)
(Eurofins, 5′-FAM-CGGCCTTTAACTCC-MGBEQ-3′)
MGB probe O7
(SEQ. ID No: 9)
(Eurofins, 5′-TexasRed-CCTCCTAGTTCCCC-MGBEQ-3′)
MGB probe N6
(SEQ. ID No: 10)
(Eurofins, 5′-NED-ACCTACCGGCCTCC-MGBEQ-3′)

The adhesive foil was removed from the incubated sample plate carefully and 36 μl of PBS was added to the V-bottom 96-well PCR Sample Plate containing the binding reaction (represents the first 20-fold dilution). Mixed vigorously by pipetting up and down 30 times. To target a lambda of 0.15 for the antibodies in the final dPCR additional dilution steps in PBS were carried out. The usual dilution factor for an antibody concentration of 8×10−11 M (ABX) is about 5,780-fold including the 20 fold dilution in previous step, so a usual dilution step transfers a μl sized volume and dilute it usually up to a few hundred times in a step (a dilution of 289 fold still needs to be made in this example), however smaller dilution steps also can be applied. To maintain precision, it is recommended to use a high precision, calibrated, electronic, conveniently multichannel, pipette with mixing capabilities. Vigorously mixing by pipetting up and down 30 times is mandatory. The dilution made by the Qiacuity mastermix was not considered as a part of the dilution aboves, however the standard unit for dPCR results is copies per μl, which needs to be adjusted accordingly. Finally, 1 μl from the last dilution step was carried over into 41 μl QiaCuity mastermix reactions prepared previously. Mixed vigorously. The reaction is ready for dPCR. Using 42 μl volume is recommended to avoid bubble formation at loading the Qiacuity Nanoplate. 40 μl of the mastermix containing the diluted samples was transferred to the dPCR 26K 24 well plate and seal the plate according to Qiacuity user manual protocol. To carry out Qiacuity (QIAGEN) dPCR the following cycling and imaging conditions were applied: (a) priming, (b) hot-start (95° C. for 2 min), (c) cycling 40 times including denaturing (95° C. for 15 sec), annealing/extension (58° C. for 30 sec). During the run the imaging conditions were the green channel for P8 with an exposure duration of 500 ms and a gain of 6, the yellow channel for BL with an exposure time of 400 ms and a gain of 6, the Red Channel for O7 with an exposure time of 300 ms and a gain of 4 and the orange channel for N6 with an exposure time of 400 ms and a gain of 6. The evaluation needs specific software partly supplied by Qiacuity to calculate the copies number of antibodies and double positive compartments, using this data the copy number of couplexes can be calculated based on Poission statistics.

### Example 2

There are four members in the human epidermal growth factor receptor family including EGFR/HER 1/ErbB1, HER2/ErbB2, HER3/ErbB3, and HER4/ErbB4. HER1, HER2 and HER3 are overexpressed in 30-40%, 20-30% and ~20% of breast cancer cases, respectively. Pharmacological targeting of HER2 has been proven to be an effective therapeutic approach. The approval of trastuzumab in 1998 and recently pertuzumab, both are HER2 antibodies, has significantly improved the outcome of breast cancer patients and their combination delivers even better clinical results [1]. Trastuzumab and pertuzumab sensitize the cells against antibody-dependent cell-mediated cytotoxicity (ADCC) as the main anticipated therapeutic effect, but they also have a significant signaling remodeling activity [2]. Pertuzumab blocks heterodimerization of HER2 with EGFR (HER1) and HER3, while trastuzumab promotes homodimerization, and both of them influence the phosphorylation of HER2 at different residues [2]. They bind HER2 at different epitopes enabling their concurrent binding. The cryo-EM structure of HER2-trastuzumab-pertuzumab has been resolved and no cooperative interaction has been found between the antibodies [3], confirming their use as independent binding agents. Their dissociation constants (K_{d}) are also known, trastuzumab has a K_{d} of 0.52 to 0.92×10−9 (M) while pertuzumab has a Kd of 0.77 to 1.42×10−9 (M) [4]. The trastuzumab and pertuzumab are characterized binding agents in the protein interaction coupling assay to detect and quantify HER2 protein. Conveniently, the assay serves as a highly validated reference assay with known absolute results, which can be reproduced and confirmed. Absolute homogeneous internal multiplexing enables a proof of principle assay with three antibodies with a self-confirmatory three-way absolute homogeneous internal multiplexing readout using HIS-tagged recombinant HER2 as shown in FIG. 1.

### Three-Way Absolute Homogeneous Internal Multiplexing Readout HER2 Protein Interaction Coupling Assay

The assay was performed as described according to Example 1, except: 50 μg of lyophilized Recombinant Human ErbB2/Her2 His-tag Protein (R&D Systems Cat. #10126-ER-050, 70 kD) was diluted in 100 μl LBTW and vortexed vigorously for 5 minutes. The solution was diluted 1:12.5 in LBTW and dissolved at 30° C. for 15 minutes and sonicated at full-power for 5 minutes. The HER2 Stock (HER2S) has a concentration of 0.04 μg/μl (5.71×10⁻⁷ M) (according to the supplier data). The ABX was prepared using Trastuzumab (TTZ)-BL label, (Kanjinti from Amgen), Pertuzumab (PTZ)-P8 label, (Perjeta from Roche), and 6×His, His-Tag Monoclonal antibody (Anti-His)-OC label, (proteintech, Cat #66005-1-Ig) at a concentration of 8×10⁻¹¹ M for each antibody.

To set up the binding reaction, HER2S was diluted in PBS 15-, 75-, 375-, 1875-, 9375-fold (3.8×10⁻⁸, 7.6×10⁻⁹, 1.5×10⁻⁹, 3.0×10⁻¹⁰, 6.1×10⁻¹¹ M). In a 48-well PCR plate (Thermo Fischer Scientific, Cat #AB0648) and 2 μl of the dilutions of HER2S (different dilutions) and 2 μl of the ABX antibodies were combined. A 48-well PCR plate with the 4 μl mixtures was sonicated at full-power for 1 minute, spun down and sealed with an adhesive foil (Thermo Fischer Scientific, Cat #4306311). The binding reaction concentration is half of the sample concentration, so the lowest concentration measured is 3.0×10⁻¹¹ M, the expected assay sensitivity is at least 10⁻¹² M (data not shown), however by upscaling of compartments, using more wells, the sensitivity of assay can be increased significantly. The 2 μl of the samples contains 7.6×10⁻¹⁴, 1.5×10⁻¹⁴, 3.0×10⁻¹⁵, 6.0×10⁻¹⁶ and 1.2×10⁻¹⁶ mol of HER2, respectively. Regarding the amount of material, the assay sensitivity is in the low attomole range (approx. 2×10⁻¹⁸ mol), assuming the sensitivity above. However, considering the mediocre Kd of the antibodies used, and number of compartments of a single well only, further improvement is expected. Parallels of four for the binding reactions were prepared for each dilution. For ABC control, 2 μl ABC Buffer with 2 μl of ABX was combined and three parallels of ABC were prepared. For the single NTC (non-template control) 4 μl of LBTW was pipetted in. The plate was sealed with an adhesive foil, sonicated at full-power (100%) for 1 min floating in an ultrasonic bath. Plate was centrifuged briefly to collect the liquid at the bottom. Sonication helps dispersing the sample evenly ensuring unbiased partitioning of the molecules. The binding reactions were incubated overnight at 4° C. Next day, 36 μl of PBS was added using a multichannel pipette to the 48-well PCR plate containing the binding reaction (represents the first 20-fold dilution of the ABX). The solution was mixed vigorously by pipetting up and down 30 times. 1 μl of diluted binding reaction was transferred into 16 μl PBS and mixed vigorously. This step was repeated once more. Then 1 μl was transferred into 41 μl master mix and mixed five times by pipetting up and down carefully. From the binding reaction these dilution steps represent a 5,780 fold dilution. 42 μl volume of mastermix was used to avoid bubble formation on loading the QIAcuity Nanoplate. 40 μl of the mastermix was transferred containing the diluted samples to the dPCR 26K 24 well plate and the plate sealed according to QIAcuity user manual protocol. dPCR settings from Example 1 were used.

The results in FIGS. 2 and 3 show the typical calibration curves of a protein interaction coupling assay, the curve is non-bijective, as at high concentration of the HER2 target protein no couplexes form due to the lack of concurrent binding of two antibodies to the same target protein, e.g. targets compete the antibodies out. Similarly, at a low concentration of HER2, the target protein itself limits the formation of couplexes. The assay has a theoretical zero background as the partitioning is a physical process having no background assuming a fully dispersed sample. It is noteworthy that a background emerging from aspecific binding of the antibodies is also highly unlikely due the strict requirement of the couplex signal of protein interaction coupling having two antibody bindings per target. All measurements are evaluated by using a proprietary evaluation software resulting in couplex data as number of couplexes per reaction units.

ABCs are also evaluated and the determined offset of couplexes different from the zero value (the expected value of ABC) as a mean is used to normalise the data by subtracting the ABC offset from the data points of samples. Non-zero ABC indicates many possible errors, but primarily it is used to control biases during the evaluation of the dPCR data. However, at a lambda of 0.15, these error sources affect the values minimally and the ABCs are, expectedly, close to zero. The application of ABC correction is validated by the (close to) zero readout of the 15-fold diluted sample for all three readouts, as they are rendered to be zero by the high concentration of the HER2 protein target. The zero results of this sample are interpreted as an implication of the zero background of the assay and the correctness of the ABC based normalization procedure.

FIG. 4 validates the calculation method showing the calculated concentration of recombinant HER2 measured as compared to the concentration of the standard stock solution

### Example 3

Four-Way Absolute Homogeneous Internal Multiplexing Readout of HER2-HER3 Proteins and their Interactions Including Protein Interaction Coupling Assay

The assay was carried out according to Example 1, except: the breast cancer cell lines MCF7 or BT474 were cultured in Nunc™ EasYFlask™ Nunclon™ Delta Surface (Thermo Scientific™, Cat #156499) at 37° C./5% CO2. For MCF7, DMEM, high glucose, GlutaMAX™ Supplement, pyruvate (Thermo Scientific™, Cat #10569010), 10% FBS (Gibco™) and 1% Pen-Strep (Gibco™) cell culture medium was used, while for BT474, DMEM/F-12, GlutaMAX™ Supplement (Thermo Scientific™, Cat #31331028) 10% FBS (Gibco™) and 1% Pen-Strep (Gibco™) were used. When the cells were confluent, the cell culture medium was removed and the cells were washed with PBS (Gibco™, Cat #14190-094). The adherent growing cells were detached from the flask surface using a cell scraper (Merck, Cat #C5981). PBS was added to the detached cells and the cell suspension was transferred to a 50-ml Falcon tube (Corning, Cat #352070). 1×10⁶ cells were aliquoted and pelleted by centrifugation (400 rcf, 5 min). The cell pellet was resuspended in 100 μl BS3W and incubated at room temperature for 30 min. After addition of 1 ml PIC-PBS to the cell suspension, it was centrifuged (400 rcf, 5 min) and the supernatant was discarded. The washing step was repeated. 1×10⁶ cells were aliquoted and pelleted by centrifugation (400 rcf, 5 min). For cell lysis, 100 μl LBTW was added to the cell pellet, resuspended, and incubated at 4° C. for 3 h. The cell lysate was then sonicated for 5 min at full power in a sonicator bath (Avantor™, Cat #142-0083P). The lysate was transferred to a QiaShredder (Qiagen, Cat #79656) and centrifuged at 21,000 g for 2 minutes. The flow-through was diluted in a two-fold dilution series in LBTW and the dilutions were used for subsequent experiments.

Antibodies were labeled with a unique oligonucleotide using the PICOglue Antibody Labeling Kit (PICO gAL Kit, Actome, Cat. #PICO-0000110), briefly using orthogonal azide chemistry to attach two labels per antibodies at the trimmed N-glycosylated Asn-297, described elsewhere (methods for carrying out this process are known in the art, including the method described in U.S. Pat. No. 99,873,736). For the HER2/HER3 detection, Pertuzumab (PTZ) (Perjeta from Roche) was labeled with the P8 label, ErbB2 (HER-2) Monoclonal Antibody (3B5) (Invitrogen, Cat #MA5-13675) was associated with the BL label, ErbB3 Monoclonal Antibody (2F12) (Invitrogen, Cat #MA5-12675) was labeled with the N6 label and ERBB3 Monoclonal Antibody (2A4) (Invitrogen, Cat #H00002065-M03) was associated with the O7 label. Labeling efficiency was determined using the 2100 Bioanalyzer Instrument (Agilent) and antibody concentrations were determined by dPCR following the instructions in Actome's PICOglue Antibody Labeling Kit manual. Briefly the labeling efficiency was calculated from the determined concentration of the size-shifted heavy-chain peak of the antibodies. The ABX (antibody mix) contained equal concentration of all four labeled antibodies and five ABX with varying antibody concentrations were prepared. ABX #1 contained 1×10⁻⁹ M antibody concentration, ABX #2 2×10⁻¹⁰ M, ABX #3 4×10⁻¹¹ M, ABX #4 8×10⁻¹² M and ABX #5 1.6×10⁻¹² M. To carry out the four-way absolute homogeneous internal multiplexing readout for HER2-HER3 detection in MCF7 and BT474 cells, isomolar titration experiments with varied concentration of antibodies were prepared. In the wells of a 48-well PCR plate (Thermo Scientific™, Cat #AB0648), 2 μl of the MCF7 cell lysate or the 1:10 in LBTW diluted BT474 cell lysate was added to 2 μl of ABX, setting up binding reactions from ABX #1 to ABX #5, using four replicates for each combination. Four replicas of antibody control (ABC) sample were prepared by combining 2 μl of ABC buffer and 2 μl of ABX #3 in the same 48-well PCR plate. The 48-well plate with the 4 μl mixtures was placed into a sonicator bath and was sonicated at full-power for 1 minute, spun down and sealed with an adhesive foil (Thermo Scientific™, Cat #4306311) and incubated overnight at 4° C. The next day the samples were diluted in PBS.

Protein interaction coupling dPCR detection. For one QIAcuity Nanoplate 26k 24-well (Qiagen, Cat #250001) a total of 1062.5 μl mastermix is prepared. Therefore, 284 μl of QIAcuity Probe Mix (Qiagen, Cat #250101), 36.3 μl of primer mix containing 25 UM forward primer and 25 μM reverse primer, 45.4 μl of 10 μM MGB probe BL, 42 μl of 10 UM MGB probe P8, 42 μl of 10 UM MGB probe O7, 42 μl of 10 μM MGB probe N6 (all from Eurofins) and 651.4 μl DNAse- and RNAse-free H2O are mixed. The 4 μl ABX and sample are diluted with PBS to a lambda (average number of target molecules per partition) of 0.15 in 42 μl mastermix in the dPCR. The applied dilution factor is recorded for later evaluations. 40 μl mastermix with sample are loaded into the nanoplates and sealed according to the instructions of the manufacturer. The digital PCR is run in a QIAcuity One, 5plex Device (Qiagen, Cat #911021) and after priming 40 cycles are applied with a denaturing step at 95° C. for 15 seconds and an annealing step at 58° C. for 30 seconds. For imaging set-up the green channel for P8 with an exposure duration of 500 ms and a gain of 6, the yellow channel for BL with an exposure time of 400 ms and a gain of 6, the Red Channel for O7 with an exposure time of 300 ms and a gain of 4 and the orange channel for N6 with an exposure time of 400 ms and a gain of 6.

The dissociation constant (Kd) of the antibodies were determined according to WO2020260277A1. Briefly using an isomolar titration experiment, the same antigen concentration was measured at different concentration of antibodies (ABX) deriving the measured antigen concentrations (MACs) for all ABX experiments choosing Kds to minimize the standard deviation of the MACs. The concentration of couplexes were calculated in the binding reaction taking into account the labeling efficacy (from Bioanalyser data) compensating for the unlabeled fraction of the antibodies (unlabeled fraction has no dPCR signal). The couplexes were ABC normalised compensating the clustering biases introduced at the evaluation of the dPCR data (ABC needs to have zero readings based on the theory, if not this is compensated in all samples equally), and taking into account of the dilution made after the binding reaction step. To get the valid concentration of couplexes and the antibodies it was assumed that the dPCR has no background regarding both the counts of antibodies and also counts of couplexes measured, and there are no failed detections, both assumptions are based on the literature [6] and mathematical proofs (disclosed elsewhere). MACs are derived using equations described in WO2020260277A1 taking Kds of antibodies, concentration of couplexes, and concentration of antibodies as input parameters.

The calibration curves of HER2-HER3 of 2000 of BT474 cells (1:10 dilution) are shown in FIG. 5. Pertuzumab (PTZ) was labeled with the P8 label (green—G), ErbB2 3B5 was associated with the BL label (yellow—Y), ErbB3 2F12 was labeled with the N6 label (orange-O) and ERBB3 2A4 was associated with the O7 label (red—R). ABC correction was applied. Samples #1-#5 are indicated with the dilution compensated antigen concentrations. The legend indicates the color pair GY—green-yellow, GR—green-red, YR—yellow-red. Y axis is molar couplex concentration, X axis is analyte (HER2m HER2:HER3 interaction and HER3, depending the antibody pairs measured) concentration, both are logarithmic. The horizontal dotted line is the calculated LOD (limit of detection).

The calibration curves of HER2-HER3 of 20,000 of MCF7 cells (no dilution) are shown in FIG. 6. Pertuzumab (PTZ) was labeled with the P8 label (green—G), ErbB2 3B5 was associated with the BL label (yellow—Y), ErbB3 2F12 was labeled with the N6 label (orange—O) and ERBB3 2A4 was associated with the O7 label (red—R). ABC correction was applied. Samples #1-#5 are indicated with the dilution compensated antigen concentrations. The legend indicates the color pair GY—green-yellow, GR—green-red, YR—yellow-red. Y axis is molar couplex concentration, X axis is analyte (HER2, HER2:HER3 interaction and HER3, depending the antibody pairs measured) concentration, both are logarithmic. The horizontal dotted line is the calculated LOD (limit of detection).

HER2, HER3 and their interaction using BT474 and MCF7 cells was measured and absolutely quantified as above (FIG. 7). Absolute copies of analytes are given per cell, per antibody pairs, where GY measures the HER2 protein copies per cells, GR, GO, YO and YR measures the HER2:HER3 protein interaction copies per cells and finally OR measures the HER3 protein copies per cells. Please note that PTZ (pertuzumab-green—G) has very limited or no access to cross-linked HER2:HER3 complex and other epitope steric hindering also occurs.

All measurements are evaluated by using a proprietary evaluation software resulting in couplex data as number of couplexes per reaction units. ABCs are also evaluated and the determined offset of couplexes different from the zero value (the expected value of ABC) as a mean is used to normalise the data by subtracting the ABC offset from the data points of samples. Non-zero ABC indicates many possible errors, but primarily it is used to control biases during the evaluation of the dPCR data. However, at a lambda of 0.15, these error sources affect the values minimally and the ABCs, expectedly, are close to zero.

The structure of the HER2/HER3 complex, their interaction with each other and the antibodies using BT474 is depicted in FIG. 8.

The four-way absolute homogeneous internal multiplexing readout evaluation enables complex and concise understanding of the biological system under investigation. The following reasoning exemplifies the possibilities. The pertuzumab (PTZ) has limited access to the cross-linked HER2:HER3 complex, so as a consequence all the measurements involving PTZ are affected rendering GR and GO measurements very low or zero and GY is measuring mainly the free HER2 (excluding the interacting amount of HER2). This behaviour is expected on the basis of literature [2]. On the basis of this data it can be suggested that the PTZ epitope on the interacting HER2 has an accessible and a blocked conformation which are differentially detected by PTZ (see GO in both cells lines), but this assumption need to be validated. It was assumed that the YO pair measures the full amount of HER2:HER3 interaction, as a consequence YR pair measurement is also affected by steric hindrances (otherwise they need to be equal), which effect is reproduced in both BT474 and MCF7 cells and as the HER3 (OR) level is less than the YO signal, it can be concluded that the red (R) antibody (2A4) is affected (as its signal is below the signal of YO). This possibly indicates that there is a third interacting protein, which is interacting with HER3 and blocks the 2A4 epitope. To validate these assumptions and get the full amount of HER2 and HER3 further experiments can be devised including non-crosslinked version of this experiment or using other antibodies instead of PTZ and 2A4 or detecting homodimers of both HER2 and HER3 using an antibody with sterically accessible epitopes labeled separately with two different labels (expecting 50% of homodimer detections). The gained information enables better functional studies of the biological system in question and can be exploited as diagnostic concepts, as well.

### Example 4

### Four-Way Absolute Homogeneous Internal Multiplexing Readout/Three-Way Absolute Homogeneous Internal Multiplexing Readout of 4EBP1-p4EBP1 Protein Modification Coupling Protein Assay

4EBP1 encodes a member of a family of translation repressor proteins as the protein binds directly to the eukaryotic translation initiation factor 4E (elF4E), and limits the assembly of the cap binding complex at the 5′ end of mRNAs. This protein is phosphorylated at many residues in response to various signals leading to its dissociation from elF4E and activation of mRNA translation. [5]

The experimental conditions were as described in Example 1, except: acute myeloid leukemia (AML) cell line U937 were cultured in Nunc™ Non-treated Flasks (Thermo Scientific™, Cat #156800) in RPMI 1640 medium, GlutaMAX™ supplement (Thermo Scientific™, Cat #61870044), 10% FBS (Gibco™) and 1% Pen-Strep (Gibco™) at 37° C./5% CO₂. For the 4eBP1-P detection, Phospho-4EBP1 (Thr37, Thr46) Recombinant Rabbit Monoclonal Antibody (4EB1T37T46-A5) (Invitrogen, Cat #MA5-27999) was labeled with the P8 label, 4EBP1 Monoclonal Antibody (554R16) (Invitrogen, Cat #AHO1382) was associated with the BL label, EIF4EBP1 monoclonal antibody (M01), clone 4F3-H2 (Abnova, Cat #H00001978-M01) was labeled with the N6 label and 6*His, His-Tag Monoclonal antibody (Proteintech, Cat #66005-1-Ig) was associated with the O7 label. 4EBP1-p4EBP1 detection in U937 calibration curve experiment. The U937 cell lysate was diluted in LBTW 2-fold. In the wells of a 48-well PCR plate (Thermo Scientific™, Cat #AB0648) 2 μl of the diluted cell lysate and 2 μl of the ABX #3 (4×10⁻¹¹ M) were added. For each sample replicas of four were applied. The 48-well plate with the 4 μl mixtures was placed into a sonicator bath and was sonicated at full-power for 1 minute, spun down and sealed with an adhesive foil (Thermo Scientific™, Cat #4306311) and incubated overnight at 4° C. The next day the samples were diluted in PBS.

The absolute quantitative Four-way absolute homogeneous internal multiplexing readout/three-way absolute homogeneous internal multiplexing readout analysis (FIG. 9) allows confirmatory and specificity control measurements to be performed in parallel and subjecting the same protein molecules as targets, making these measurements independent yet highly concordant. The following reasoning exemplifies the possibilities. The gained information enables better functional studies of the biological system in question and can be exploited as diagnostic concepts, as well. The YO pair measures the amount of the target protein regardless of the phosphorylation status, setting an upper bound for the amount of the phosphorylated protein. GY and GO measure the phosphorylated amount via two independent antibody pairs that form a pair of confirmatory reactions. As a conclusion, the 4EBP1 is highly and concordantly phosphorylated. Finally, the fourth antibody (red) targeting a non-specific target, a HIS tag, serves as general specificity measure for all of the antibodies applied, and together with the zero background the assay (supported by independent experimental and mathematical proofs) proves the absence of the non-specific cross-reactions of the antibodies used.

### Example 5

### Antibody Mix Preparation (ABX)

Two anti-4EBP1 antibodies and one anti-Phospho 4EBP1 antibody were labelled with an oligonucleotide (PICOglue, Actome). Anti-4EBP1 4F3-H2 (Cat #H00001978-M01, Thermofisher Scientific) was labelled with the 07 (Actome) label, Anti-4EBP1 2C3F3 (Cat #60246-1-Ig, Proteintech) with the BL (Actome) label and Anti-Phospho 4EBP1 (Cat #MA5-36935, Thermofisher Scientific) with the P8 (Actome) label. The labelled were mixed in ABC buffer at equimolar concentration, so that in the binding reaction with the single cell they reach a concentration of 5.5×10⁻¹¹ M and 0.5 M Fluorescein (Cat #97062-186, VWR) was added. The cell lysate was treated with λ-phosphatase (PPase or left untreated (control). 2 uL of labelled antibody mixture was added to 2 μl cell lysate (in LBTW lysis buffer) (20000 cell), mixed and incubated overnight at 4 C.

dPCR (QIAcuity)

For one QIAcuity Nanoplate 26k 24-well (Qiagen, Cat #250001) a total of 1050 μl Mastermix are prepared. Therefore, 262.5 μl of QIAcuity Probe Mix (Qiagen, Cat #250101), 33.6 μl of Actome Primer Mix containing Actome primers and BLA, P8 O7 probes, and 602.9 μl DNAse- and RNAse-free H2O are mixed. The samples are diluted with PBS to a lambda (average number of target molecules per partition) of 0.15 in 42 μl mastermix in the dPCR. 40 μl mastermix with sample are loaded into the nanoplates and sealed according to the instructions of the manufacturer. The digital PCR is run in a QIAcuity, 5 plex device (Qiagen) and after priming 40 cycles are applied with a denaturing step at 95° C. for 15 seconds and an annealing step at 58° C. for 30 seconds. For imaging set-up the Green Channel for P8 with an exposure duration of 500 ms and a Gain of 6, the Yellow Channel for BLA with an exposure time of 400 ms and a Gain of 6, the Red Channel for O7 with an exposure time of 300 ms and a gain of 4. All Actome reagents available from Actome, Freiburg.

The results of the dPCR were evaluated for the couplexes using Actome's AMULATOR software, allowing for absolute quantification of the proteoforms. 100 k 4EBP1 protein was expressed per cell and the proteins were up to 100% phosphorylated. All these measurements are done parallel in a single PICO reaction (16 replicates). Triangular PICO measurements allow a self confirmatory protein phosphorylation measures.

As shown in FIG. 11 untreated samples showed comparable 4EBP1 and p-4EBP1 absolute quantitative amounts, while λ-phosphatase treatment significantly reduced the p-4EBP1 signal, as expected.

### LITERATURE CITED

1. Nami B, Maadi H, Wang Z. Mechanisms underlying the action and synergism of trastuzumab and pertuzumab in targeting HER2-positive breast cancer. Cancers (Basel). 2018; 10 (10). doi: 10.3390/cancers10100342
2. Nami B, Maadi H, Wang Z. The effects of pertuzumab and its combination with trastuzumab on HER2 homodimerization and phosphorylation. Cancers (Basel). 2019; 11 (3): 1-20. doi: 10.3390/cancers11030375
3. Hao Y, Yu X, Bai Y, Mcbride H J, Id X H. Cryo-EM Structure of HER2-trastuzumab-pertuzumab complex. 2019:1-10.
4. Lua W H, Ling W L, Yeo J Y, Poh J J, Lane D P, Gan S K E. The effects of Antibody Engineering CH and CL in Trastuzumab and Pertuzumab recombinant models: Impact on antibody production and antigen-binding. Sci Rep. 2018; 8 (1): 1-10. doi: 10.1038/s41598-017-18892-9\
5. Pause A., Belsham G. J., Gingras A. C., Donze O., Lin T. A., Lawrence J. C., Jr, Sonenberg N. Insulin-dependent stimulation of protein synthesis by phosphorylation of a regulator of 5′-cap function. Nature. 1994; 371:762.
6. Quan, P. L., Sauzade, M., & Brouzes, E. (2018). DPCR: A technology review. Sensors (Switzerland), 18 (4). https://doi.org/10.3390/s18041271

1. A method of determining the concentration and/or status of an analyte comprising:
(a) contacting the analyte with three or more binding agents, wherein each of said binding agents binds to a different site on the analyte and is associated with a unique label;
(b) detecting the amount of analyte bound by two or more pairs of binding agents using a bi-component detection method;
(c) determining the concentration and/or status of the analyte present based on the measurements obtain in step (b).

2. The method of claim 1 wherein the binding agents are each independently selected from nucleic acids, antibodies, peptides, proteins, aptamers, molecularly imprinted polymers, cells or combinations thereof.

3. The method of claim 1, wherein at least one of the binding agents is a monoclonal antibody.

4. The method of claim 1, wherein one of the binding agents binds specifically to an unmodified or modified analyte.

5. The method of claim 4 wherein the modified analyte has been modified by phosphorylation, methylation, sulfation, acetylation, ubiquitylation, prenylation, myristoylation, sumoylation, palmitoylation, different types of glycosylation (N-glycosylation, O-glycosylation, C-glycosylation and S-glycosylation), phosphoglycosylation, glycosylphosphatidylinositol (GPI anchored), methylation or other known amino acid or protein modification.

6. The method of claim 1, wherein the analyte is a protein, peptide, nucleic acid, carbohydrate, lipid, microorganism or fragment thereof, cell, or complex or combinations thereof.

7. A kit for determining the concentration and/or status of an analyte comprising:
(a) three or more labelled binding agents, wherein each of said binding agents binds to a different site on the analyte suitable for use in bi-component detection method; and
(b) reagents for detecting pairs of binding agents using a bi-component detection method; and/or
(c) a computer programme which when run on a computer analyses the results of the bi-component detection method and determines the concentration of the analyte in a sample; and optionally
(d) instructions for carrying out the bi-component detection method.

8. The kit of claim 7 wherein the reagents for detecting pairs of binding agents using a bi-component detection method, wherein the binding agents are each labelled with an unique oligonucleotide sequence, comprises
(i) a forward primer and reverse primer for amplifying the unique oligonucleotide sequences; and
(ii) three or more oligonucleotide probes, where each oligonucleotide probe is independently capable of specifically binding to a unique oligonucleotide sequence amplified by the primers and independently labelled
